(19) United States
(12) Patent Application Publication
(10) Pub. No.: US 2026/0224150 A1
(43) Pub. Date: Aug. 6, 2026

(54) BIOLOGICAL INFORMATION MEASUREMENT DEVICE, CONTROL METHOD OF BIOLOGICAL INFORMATION MEASUREMENT DEVICE, INFORMATION PROCESSING SYSTEM, AND STORAGE MEDIUM

(71) Applicant: OMRON HEALTHCARE CO., LTD., Muko-shi (JP)

(72) Inventors: Kentaro MORI, Kyoto (JP); Tsuyoshi Kitagawa, Kyoto (JP)

(73) Assignee: OMRON HEALTHCARE CO., LTD., Muko-shi (JP)

(21) Appl. No.: 19/445,889

(22) Filed: Jan. 12, 2026

(30) Foreign Application Priority Data
Feb. 5, 2025 (JP) 2025-017924

(51) Int. Cl. A61B 5/339; A61B 5/00; A61B 5/349; G16H 50/30

(52) U.S. Cl. CPC A61B 5/339; A61B 5/349; A61B 5/746; G16H 50/30

(57) ABSTRACT

A biological information measurement device that acquires a biological signal and calculates a measurement value for a predetermined index related to the biological signal on the basis of the biological signal acquired, the biological information measurement device including: an arrhythmia information acquisition unit that acquires information related to presence or absence of arrhythmia during acquisition of the biological signal; a display unit including an area capable of displaying the measurement value; an input unit; and a control unit, wherein the control unit calculates the measurement value on the basis of the biological signal acquired, and causes, in a case where the information acquired by the arrhythmia information acquisition unit indicates that arrhythmia during the acquisition of the biological signal has been present, the display unit not to display the measurement value calculated but to display information of a fact that arrhythmia has been present.

### CROSS-REFERENCE TO RELATED APPLICATIONS

The disclosure of Japanese Patent Application No.JP 2025-017924, filed on February 5, 2025 including the specification, drawings and abstract is incorporated herein by reference in its entirety.

### TECHNICAL FIELD

The present invention relates to a biological information measurement device, a control method of a biological information measurement device, an information processing system, and a program.

### BACKGROUND ART

In recent years, it has become widespread to perform health management by measuring information regarding the body and health of an individual such as a blood pressure value and a pulse rate with a measuring instrument and recording and analyzing the measurement result. In particular, since arrhythmia such as atrial fibrillation (AF) may lead to a brain/cardiovascular disease, it is desirable that the user himself/herself can easily recognize such an abnormality.

In this regard, for example, there has been proposed a sphygmomanometer that displays, in a case where atrial fibrillation has been detected at the time of blood pressure measurement, the fact that there has been atrial fibrillation together with a blood pressure measurement result (for example, Patent Document 1). According to such a sphygmomanometer, the user can quickly recognize the presence of arrhythmia while performing daily health management of blood pressure measurement.

Furthermore, Patent Document 2 that discloses a biological information acquisition device discloses that, in a case where arrhythmia has been detected in an electrocardiogram test performed prior to blood pressure measurement, notifying the detection of arrhythmia makes it possible to notify the user of a decrease in reliability of the blood pressure measurement.

### PRIOR ART DOCUMENTS

### Patent Documents

Patent Document 1: JP 2024-039189 A

Patent Document 2: JP 2008-168074 A

### SUMMARY

However, if the measurement value is displayed as the measurement result even in a case where arrhythmia occurs, the user may focus on the displayed value and overlook the occurrence of arrhythmia. In a case where arrhythmia has been detected at the time of acquisition of the biological signal, the calculated measurement value is not accurate. Therefore, the user erroneously recognizes the inaccurate measurement value as a result of appropriate measurement in a case where the information regarding the occurrence of arrhythmia is overlooked.

In view of the above circumstances, an object of the present invention is to provide a technology capable of preventing an inaccurate measurement value from being indicated to a user and of notifying the user of the arrhythmia detected during acquisition of the biological signal in a case where arrhythmia has been present during measurement of biological information.

In order to solve the above problem, the present invention adopts the following configuration. The configuration is

a biological information measurement device that acquires a biological signal and calculates a measurement value for a predetermined index related to the biological signal on the basis of the biological signal acquired, the biological information measurement device including:

an arrhythmia information acquisition unit that acquires information related to presence or absence of arrhythmia in a living body during acquisition of the biological signal;

a display unit including an area capable of displaying at least the measurement value;

an input unit that receives an operation input by a user; and

a control unit, wherein

the control unit

calculates the measurement value on the basis of the biological signal acquired, and

causes, in a case where the information acquired by the arrhythmia information acquisition unit indicates that arrhythmia during the acquisition of the biological signal has been present, the display unit not to display the measurement value calculated but to display information of a fact that arrhythmia has been present.

Note that the information related to the presence or absence of arrhythmia acquired by the arrhythmia information acquisition unit may be information on the presence or absence of arrhythmia detected by another biological information measurement device or a biological signal itself measured by another device. Examples of “arrhythmia” include atrial fibrillation, atrial flutter, and premature contraction. The measurement value can be, for example, a blood pressure value, a pulse rate, or the like.

According to such a configuration, in a case where arrhythmia has been present during the acquisition of the biological signal, only the fact that arrhythmia has been present is displayed without displaying the measurement value. Therefore, it is possible to prevent the inaccurate measurement result from being indicated to the user and to notify the user of the arrhythmia detected during the measurement.

The biological information measurement device may further include a storage unit that holds information related to calculation of the measurement value performed in a past, the information including information related to a date and time of the calculation and presence or absence of arrhythmia during acquisition of the biological signal at the date and time, wherein

the control unit

may receive, via the input unit, a request to display, on the display unit, the information related to the measurement value of the past held in the storage unit, and

may cause, in a case where the information related to the measurement value related to the request to display includes information of the fact that arrhythmia has been present during the acquisition of the biological signal related to the calculation of the measurement value, the display unit not to display the measurement value of the past but to display the information of the fact that arrhythmia has been present during the acquisition of the biological signal.

According to such a configuration, the user can view the past measurement history, and even in a case where arrhythmia has been detected at the time of the past measurement, it is possible to prevent an inaccurate measurement result from being indicated to the user, and to notify the user of the arrhythmia detected during the acquisition of the biological signal.

Furthermore, the control unit

may calculate a first average value that is an average value of the measurement values in a case where the calculation of the measurement value is performed a plurality of times at predetermined intervals according to an operation by a user, and may cause the display unit to display the first average value, and

may cause, in a case where the information acquired by the arrhythmia information acquisition unit indicates that arrhythmia has been present during the acquisition of the biological signal in any of the plurality of calculations of the measurement value, the display unit not to display the first average value but to display information of the fact that arrhythmia has been present.

For example, in a case where blood pressure or the like is measured in the morning and the night, it is desirable from the viewpoint of health management that measurement is performed a plurality of times at short intervals and an average value thereof is used as a measurement value in one measurement opportunity, because the measurement values show less variation as compared with a case where measurement is performed only once in each measurement opportunity. According to the configuration as described above, even in a case where the measurement value of one measurement opportunity is provided using the average value of measurement values from such plurality of times of measurement, it is possible to prevent an inaccurate measurement result from being indicated to the user and to promptly notify the user of arrhythmia.

In the present specification, terms such as first and second are used to describe various components, but the terms are used only for the purpose of distinguishing one component from other components, and do not mean any hierarchy or order. For example, the first component can be referred to as a second component without departing from the scope of rights of the present invention, and the second component can also be referred to as a first component.

Furthermore, the control unit

may receive, via the input unit, a request to calculate a second average value that is an average value in past any period using the information related to the calculation of the measurement value held in the storage unit,

may exclude, in a case where any of a plurality of pieces of the information related to the calculation of the measurement value related to the request to calculate includes information of the fact that arrhythmia has been present during the acquisition of the biological signal, the information related to the calculation of the measurement value including the information of the fact that arrhythmia has been present to calculate the second average value, and

may cause the display unit to display the second average value calculated.

According to such a configuration, the user can view the average value for each time zone (for example, measurement opportunity at morning/night) in past any period (for example, last one week), and it is possible to prevent the average value from being calculated using the inaccurate measurement value of the case where arrhythmia has been detected.

Furthermore, the biological information measurement device may further include a pulse wave acquisition unit that acquires a pulse wave of the living body, wherein

the arrhythmia information acquisition unit may acquire the information related to presence or absence of arrhythmia in the living body on the basis of the pulse wave.

Furthermore, the biological information measurement device may further include an electrocardiographic waveform acquisition unit that acquires an electrocardiographic waveform of the living body, wherein the arrhythmia information acquisition unit may acquire the information related to presence or absence of arrhythmia in the living body on the basis of the electrocardiographic waveform.

Furthermore, the control unit may cause, in a case where a user operation to display a measurement value calculated when the arrhythmia has been present is received via the input unit after causing the display unit not to display the measurement value but to display information of the fact that arrhythmia has been present, the display unit to display the measurement value.

According to such a configuration, the user can recognize the occurrence of arrhythmia, and, based on the fact (that is, on the premise that the measurement value is inaccurate), can refer to the blood pressure value measured.

Furthermore, the present invention can also be regarded as a method as follows. The method is

a control method of a biological information measurement device, the control method including:

acquiring a biological signal;

calculating a measurement value for a predetermined index related to the biological signal on the basis of the biological signal;

determining presence or absence of arrhythmia in the living body during acquisition of the biological signal; and

displaying a measurement result related to the index, wherein,

in a case where it is determined, in the determining presence or absence of arrhythmia, that arrhythmia has been present, a value of the measurement value calculated is not displayed but information of a fact that arrhythmia has been present is displayed in the displaying the measurement result.

Furthermore, the present invention can also be regarded as a program for causing the biological information measurement device to execute the above-described control method, and a computer-readable recording medium in which such a program is non-transiently recorded.

Furthermore, the present invention can also be regarded as a system as follows. The system is an information processing system including:

a biological information measurement device that acquires a biological signal of a living body and calculates a measurement value for a predetermined index related to the biological signal on the basis of the biological signal acquired; and an information processing terminal communicatively connected to the biological information measurement device, wherein

the information processing terminal

receives, from the biological information measurement device, at least determination information for determining presence or absence of arrhythmia in the living body during acquisition of the biological signal,

determines presence or absence of arrhythmia during the acquisition of the biological signal on the basis of the determination information, and

transmits, in a case where it is determined that arrhythmia has been present during the acquisition of the biological signal, information of a fact that arrhythmia has been present during the acquisition of the biological signal to the biological information measurement device, and wherein

the biological information measurement device includes a first display unit, and

causes, in a case where the information of the fact that arrhythmia has been present during the acquisition of the biological signal is received from the information processing terminal, the first display unit not to display the measurement value calculated but to display the information of the fact that arrhythmia has been present.

According to such a configuration, the calculation load of the biological information measurement device can be reduced, and even if a high-performance calculation device is not used, it is possible to prevent an inaccurate measurement result from being indicated to the user and to notify the user of the arrhythmia detected during the measurement.

Furthermore, the biological information measurement device may include an electrocardiographic waveform acquisition unit that acquires an electrocardiographic waveform of the living body, and the determination information may be the electrocardiographic waveform.

Furthermore, the information processing terminal may include a second display unit,

may receive the measurement value calculated by the biological information measurement device, and

may display the measurement value received on the second display unit in a case where it is determined that arrhythmia has been absent during the acquisition of the biological signal, and may not display the measurement value but may display the information of the fact that arrhythmia has been present on the second display unit in a case where it is determined that arrhythmia has been present during the acquisition of the biological signal.

Furthermore, the information processing terminal may include a second display unit,

may receive the measurement value calculated by the biological information measurement device,

may display the measurement value received on the second display unit in a case where it is determined that arrhythmia has been absent during the acquisition of the biological signal, and may not display the measurement value but may display the information of the fact that arrhythmia has been present on the second display unit in a case where it is determined that arrhythmia has been present during the acquisition of the biological signal, and

may display, in a case where a user operation to display the measurement value calculated when the arrhythmia has been present is received after causing the second display unit to display the information of the fact that arrhythmia has been present, the measurement value on the second display unit.

With such a configuration, the user can refer to the measurement result with an information processing terminal at hand such as a smartphone, and can recognize the presence of arrhythmia without looking at an inaccurate measurement value in a case where arrhythmia has been detected during the acquisition of the biological signal.

Furthermore, the present invention can also be regarded as a program for causing the information processing device to function as the above-described information processing terminals, and a computer-readable recording medium in which such a program is non-transiently recorded.

Note that each of configurations and processing described above can be combined with each other to constitute the present invention as long as no technical contradiction occurs.

According to the present invention, in a case where arrhythmia has been present during measurement of biological information, it is possible to prevent an inaccurate measurement value from being indicated to a user and to notify the user of the arrhythmia detected during acquisition of the biological signal.

### BRIEF DESCRIPTION OF THE DRAWINGS

FIG. 1 is a block diagram schematically illustrating a device configuration of a biological information measurement device according to a first embodiment;

FIG. 2 is a block diagram illustrating functional modules implemented by a control unit of the biological information measurement device according to the first embodiment;

FIG. 3 is a flowchart illustrating a flow of blood pressure measurement processing by the biological information measurement device according to the first embodiment;

FIG. 4A is a first diagram illustrating an example of a measurement result display screen when arrhythmia has been detected during blood pressure measurement in the first embodiment;

FIG. 4B is a second diagram illustrating an example of the measurement result display screen when arrhythmia has been detected during blood pressure measurement in the first embodiment;

FIG. 5 is a flowchart illustrating a flow of processing when a history viewing mode is executed in the blood pressure measurement device according to the first embodiment;

FIG. 6 is a flowchart illustrating a flow of processing when blood pressure measurement is performed in a continuous measurement mode in the blood pressure measurement device according to the first embodiment;

FIG. 7 is a first diagram illustrating an example of a measurement result display screen when the blood pressure measurement is performed in the continuous measurement mode;

FIG. 8 is a second diagram illustrating an example of the measurement result display screen when the blood pressure measurement is performed in the continuous measurement mode;

FIG. 9 is a flowchart illustrating a flow of processing when a past average viewing mode is executed in the blood pressure measurement device according to the first embodiment;

FIG. 10 is a diagram illustrating an example of the measurement result display screen when the blood pressure measurement is performed in the past average viewing mode;

FIG. 11 is a block diagram schematically illustrating a device configuration of a biological information measurement device according to a second embodiment;

FIG. 12 is a block diagram illustrating functional modules implemented by a control unit of the biological information measurement device according to the second embodiment;

FIG. 13 is a block diagram schematically illustrating a device configuration of a biological information measurement device according to a third embodiment;

FIG. 14 is a block diagram illustrating functional modules implemented by a control unit of the biological information measurement device according to the third embodiment;

FIG. 15 is a flowchart illustrating a flow of biological information measurement processing by the biological information measurement device according to the third embodiment;

FIG. 16A is a first diagram illustrating an example of the measurement result display screen when arrhythmia has not been detected during biological signal acquisition in the third embodiment;

FIG. 16B is a second diagram illustrating an example of the measurement result display screen when arrhythmia has been detected during biological signal acquisition in the third embodiment;

FIG. 17 is a block diagram schematically illustrating a device configuration of a biological information measurement device according to a fourth embodiment;

FIG. 18 is a block diagram illustrating functional modules implemented by a control unit of the biological information measurement device according to the fourth embodiment;

FIG. 19 is a flowchart illustrating a flow of biological information measurement processing by the biological information measurement device according to the fourth embodiment;

FIG. 20A is a first diagram illustrating an example of the measurement result display screen when arrhythmia has not been detected during biological signal acquisition in the fourth embodiment;

FIG. 20B is a second diagram illustrating an example of the measurement result display screen when arrhythmia has been detected during biological signal acquisition in the fourth embodiment;

FIG. 21 is a block diagram schematically illustrating an information processing system according to a fifth embodiment;

FIG. 22A is a block diagram illustrating functional modules implemented

by a control unit of the biological information measurement device according to the fifth embodiment;

FIG. 22B is a block diagram illustrating functional modules implemented by a control unit of an information processing terminal according to the fifth embodiment;

FIG. 23 is a first flowchart illustrating a flow of processing of each of the biological information measurement device and the information processing terminal in the information processing system according to the fifth embodiment; and

FIG. 24 is a second flowchart illustrating a flow of processing of each of the biological information measurement device and the information processing terminal in the information processing system according to the fifth embodiment.

### DESCRIPTION OF THE EMBODIMENTS

### FIRST EMBODIMENT

Hereinafter, specific embodiments of the present invention will be described on the basis of the drawings. However, the specific detail of each configuration described in the embodiments described below, the arrangement relationship thereof, and the like are not intended to limit the scope of the present invention only to them unless otherwise specified.

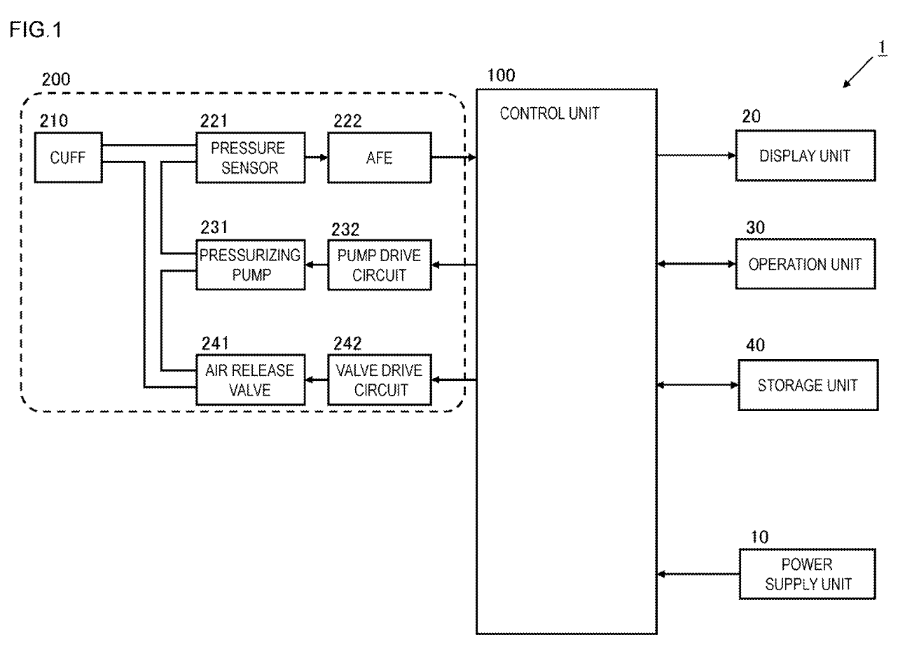

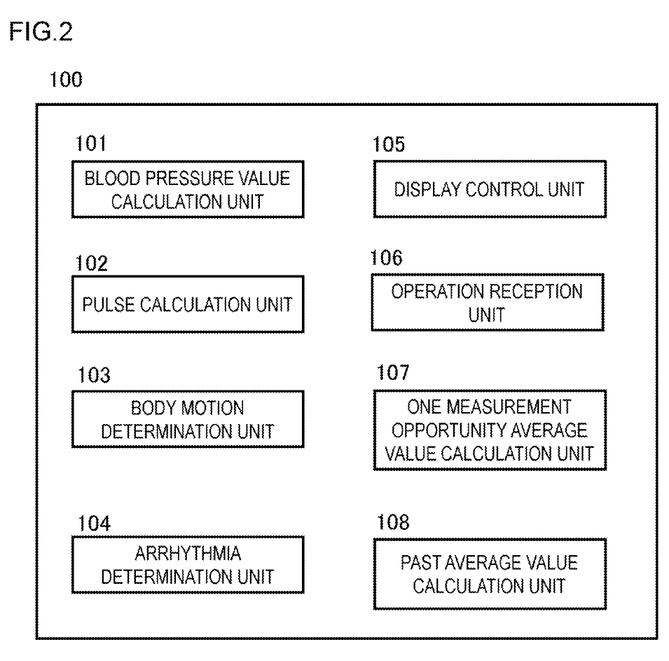

The present invention can be applied to, for example, a biological information measurement device 1 as illustrated in FIGS. 1 and 2. The biological information measurement device 1 is a so-called sphygmomanometer, and is configured to be able to measure a blood pressure value and a pulse rate. FIG. 1 is a block diagram schematically illustrating a device configuration of the biological information measurement device 1 in the present embodiment, and FIG. 2 is a block diagram illustrating an example of functional modules implemented by a control unit 100 of the biological information measurement device 1.

As illustrated in FIG. 1, the biological information measurement device 1 according to the present embodiment schematically includes components such as a power supply unit 10, a display unit 20, an operation unit 30, a storage unit 40, the control unit 100, and a blood pressure measurement unit 200, and has a configuration similar to that of a general portable upper-arm sphygmomanometer including a main body housing, a cuff, and an air tube as an appearance although not illustrated.

The power supply unit 10 is a power source of the biological information measurement device 1, and may be, for example, a rechargeable secondary battery such as a lithium ion battery, but is not limited thereto, and may be a primary battery, a terminal connectable to an external power supply, or the like.

The display unit 20 is a functional unit for displaying various types of information, and can be, for example, a display device such as a liquid crystal display (LCD). Furthermore, the operation unit 30 is a functional unit that receives a user operation such as turning on/off of a power supply, execution of measurement, and selection/decision of a menu, and can be configured by an operation switch and the like. Note that a touch panel display can also be adopted as a configuration that serves as both the display unit 20 and the operation unit 30.

The storage unit 40 includes a main storage device such as a random access memory (RAM) and a read only memory (ROM), and an auxiliary storage device such as an HDD and a flash memory, and stores various types of information such as an application program, measurement results such as a blood pressure value and a pulse, information on the presence or absence of arrhythmia to be described later, and other acquired biological information. Note that the measurement results of the blood pressure and the like and other acquired biological information may be stored in the storage unit 40 in association with time information such as the measurement (acquisition) time. As the time information, for example, time information clocked by referring to a real time clock (RTC) of the control unit 100 to be described later can be used. Furthermore, the auxiliary storage device may be configured to be detachable from the main body housing.

The control unit 100 is a functional unit that controls the entire biological information measurement device 1, and includes, for example, a processor such as a central processing unit (CPU). When receiving a user's operation via the operation unit 30, the control unit 100 controls each component of the biological information measurement device 1 to execute various processing such as blood pressure measurement and presentation of various types of information according to a predetermined program. The predetermined program is stored in the storage unit 40 and read therefrom. As the processor, in addition to the CPU, a graphic processing unit (GPU), a field programmable grid array (FPGA), an application specific integrated circuit (ASIC), or the like can be adopted.

Furthermore, functional modules illustrated in FIG. 2, including a blood pressure value calculation unit 101, a pulse calculation unit 102, a body motion determination unit 103, an arrhythmia determination unit 104, a display control unit 105, an operation reception unit 106, a one measurement opportunity average value calculation unit 107, and a past average value calculation unit 108 are implemented by the control unit 100. These functional modules will be described in detail later.

The blood pressure measurement unit 200 includes a cuff 210, a pressure sensor 221, an analog front end (AFE) 222 including an analog-to-digital (A/D) converter, a pressurizing pump 231, a pump drive circuit 232, an air release valve 241, and a valve drive circuit 242, and is a functional unit that measures the blood pressure of the user by a so-called oscillometric method. The blood pressure measurement by the oscillometric method is a well-known technology, and thus a detailed description thereof will be omitted.

The cuff 210 is a member used with being wound around the upper arm of the user and includes the pressurizing pump 231 (pump drive circuit 232) disposed in the main body housing, an air bladder (not illustrated) communicating with the air release valve 241 (valve drive circuit 242) via an air tube (not illustrated), a belt (not illustrated) incorporating the air bladder, the pressure sensor 221 provided on the belt, and the like.

Note that the belt of the cuff 210 is provided with a fixing unit (for example, a hook-and-loop fastener) for fixing the cuff 210 to the upper arm of the user, and the cuff 210 is wound around the upper arm of the user by the belt when blood pressure measurement is performed using the biological information measurement device 1.

For example, a piezoresistive sensor including a piezoelectric element can be adopted as the pressure sensor 221, and at least a pressure pulse wave of the user is acquired. In addition, the biological information measurement device 1 according to the present embodiment calculates the blood pressure and the pulse of the user on the basis of the pressure pulse wave acquired by the pressure sensor 221, and detects the presence or absence of the body motion and arrhythmia of the user during the blood pressure measurement as described later.

At the time of blood pressure measurement, the control unit 100 controls the pressurizing pump 231 (pump drive circuit 232) and the air release valve 241 (valve drive circuit 242) to adjust the cuff pressure of the cuff 210. Specifically, control is performed such that the pressurizing pump 231 is driven to send air to the cuff 210 to inflate the cuff 210 (increase the cuff pressure). In this way, after the blood flow is once inhibited by compressing the blood vessel of the upper arm of the user, control is performed such that the pressurizing pump 231 is stopped to open the air release valve 241 and the air is gradually released from the cuff 210 to contract the cuff 210 (reduce the cuff pressure).

Next, functional modules illustrated in FIG. 2 will be described. The blood pressure value calculation unit 101 calculates the blood pressure value of the user on the basis of the pressure pulse wave acquired by the pressure sensor 221. Note that, in the present embodiment, the blood pressure value is calculated by the oscillometric method, but it is also possible to provide a microphone in the cuff 210 and calculate the blood pressure value by, for example, a Korotkoff method of detecting a Korotkoff sound.

The pulse calculation unit 102 calculates the pulse rate of the user on the basis of the pressure pulse wave (such as the peak number of the pulse wave) acquired by the pressure sensor 221. The body motion determination unit 103 determines the presence or absence and the number of times of body motion during blood pressure measurement on the basis of the pressure pulse wave (such as disturbance of pulse waveform) acquired by the pressure sensor 221.

Furthermore, the arrhythmia determination unit 104 determines (detects) the presence or absence of arrhythmia during the blood pressure measurement on the basis of the pressure pulse wave (information on the time interval between the peaks of the pulse wave, and the like) acquired by the pressure sensor 221. That is, in the present embodiment, a combination of the arrhythmia determination unit 104 and the pressure sensor 221 corresponds to an arrhythmia information acquisition unit.

The display control unit 105 controls the display unit 20 (the display contents thereof). For example, the display control unit 105 causes the display unit 20 to display menu selection, measurement results, past history data, and the like, and specific display contents will be described later.

The operation reception unit 106 receives an operation input by a user via the operation unit 30. For example, when the user presses a measurement start button, an item selection button, a decision button, or the like (each including an interface expressed on an image), the biological information measurement device 1 is controlled to perform the associated operation.

The one measurement opportunity average value calculation unit 107 calculates, in a case where blood pressure measurement is performed a plurality of times (for example, three times) at predetermined intervals (for example, two minutes), an average value of the measured blood pressure values. In the present embodiment, a “continuous measurement mode” selectable by a user operation is implemented. In a case where blood pressure measurement is performed in this mode, three times of continuous measurement are automatically performed at intervals of two minutes, and an average value of three times of measurement is a measurement result of one measurement opportunity. However, even if the normal blood pressure measurement is performed a plurality of times, in a case where the condition of the one measurement opportunity is satisfied, the one measurement opportunity average value calculation unit 107 can calculate an average value afterwards using the data of the plurality of measurement results and use the average value as the measurement result of the one measurement opportunity.

The past average value calculation unit 108 calculates an average value for each time zone (morning/night) in past any period stored in the storage unit 40. For example, the user may select the period from preset periods such as three days, one week, ten days, and one month, or may decide the period on a daily basis. Alternatively, only an average value of a preset predetermined period (for example, last one week) may be calculated.

### Flow of blood pressure measurement processing

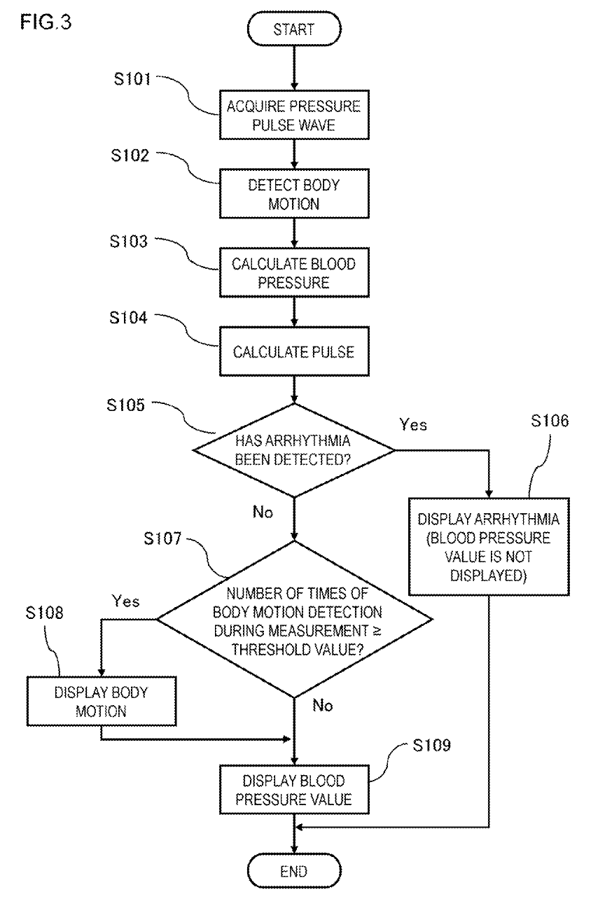

Next, a flow of processing when measurement of blood pressure is executed by the biological information measurement device 1 will be described on the basis of FIG. 3. FIG. 3 is a flowchart illustrating a flow of processing by the biological information measurement device 1 when measurement of the blood pressure is performed in a normal mode. Prior to the execution of the blood pressure measurement, the user winds the cuff 210 around his/her upper arm, and then instructs execution of the blood pressure measurement in the normal mode via the operation unit 30.

After receiving the instruction of the blood pressure measurement, the biological information measurement device 1 acquires the pressure pulse wave of the user by the blood pressure measurement unit 200 (S101). Based on the acquired pressure pulse wave, the control unit 100 (the blood pressure value calculation unit 101, the pulse calculation unit 102, and the body motion determination unit 103) detects the body motion of the user (S102), calculates the blood pressure value (S103), and calculates the pulse rate (S104). In addition, the control unit 100 (arrhythmia determination unit 104) determines the presence or absence of arrhythmia during blood pressure measurement on the basis of the acquired pressure pulse wave (S105).

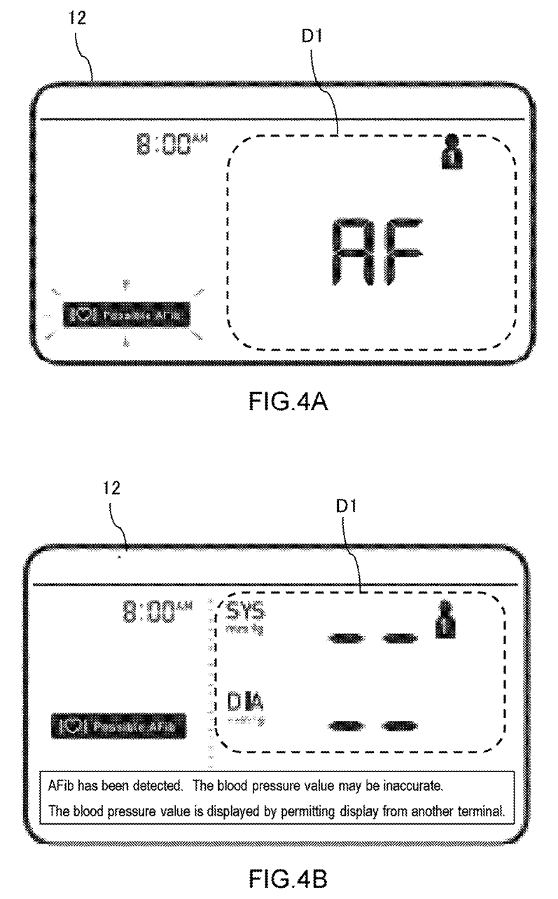

In a case where it is determined in step S105 that arrhythmia has been present during the blood pressure measurement, the control unit 100 (display control unit 105) does not display the calculated blood pressure value but displays, as a measurement result, the fact that arrhythmia has been present on the display unit 20 (S106). FIG. 4A illustrates an example of the display unit 20 when displaying the measurement result of a case where arrhythmia has been detected during the blood pressure measurement in this manner. In the example illustrated in FIG. 4A, in a blood pressure display area D1 for displaying the measured blood pressure value of the display unit 20, the blood pressure value is not displayed, but “AF” indicating that arrhythmia (in this case, atrial fibrillation) has been detected is displayed. Furthermore, a mark indicating that atrial fibrillation has been detected is also displayed in a blinking manner on the lower left side of the screen.

On the other hand, in a case where it is determined in step S105 that arrhythmia has been absent during the blood pressure measurement, the control unit 100 (body motion determination unit 103) determines whether or not the number of times of body motion detected during the blood pressure measurement exceeds a predetermined threshold value (S107). In a case where it is determined in step S107 that the number of times of body motion is equal to or less than the threshold value, the control unit 100 (display control unit 105) displays the blood pressure value in the blood pressure display area D1 of the display unit 20 (S109).

In a case where it is determined in step S107 that the number of times of body motion exceeds the threshold value, the control unit 100 (display control unit 105) displays the fact that body motion has been detected (which may result in the low accuracy of the blood pressure value) in an area other than the blood pressure display area D1 of the display unit 20 (S108), and displays the blood pressure value in the blood pressure display area D1 (S109). Note that step S108 and step S109 may be executed simultaneously.

After displaying the blood pressure measurement result on the display unit 20 in step S106 or step S109, the biological information measurement device 1 stores the measurement result in the storage unit 40 in association with the information on the measurement time, and ends the series of processing. As the information to be stored in the storage unit 40 in a case where arrhythmia has been detected during the measurement, the calculated blood pressure value may be stored in association with the information on the arrhythmia, or the calculated blood pressure value may be discarded and only the information on the arrhythmia may be stored.

With the display of the measurement result as described above, in a case where arrhythmia has been detected during the blood pressure measurement, it is possible to prevent an inaccurate blood pressure measurement result from being indicated to the user and to notify the user of the arrhythmia detected during the blood pressure measurement. Furthermore, even in a case where arrhythmia has not been detected, when the number of times of body motion exceeds the threshold value, it is possible to alert the user by displaying that the accuracy of the calculated blood pressure value has decreased together with the blood pressure value.

### Flow of past data display processing

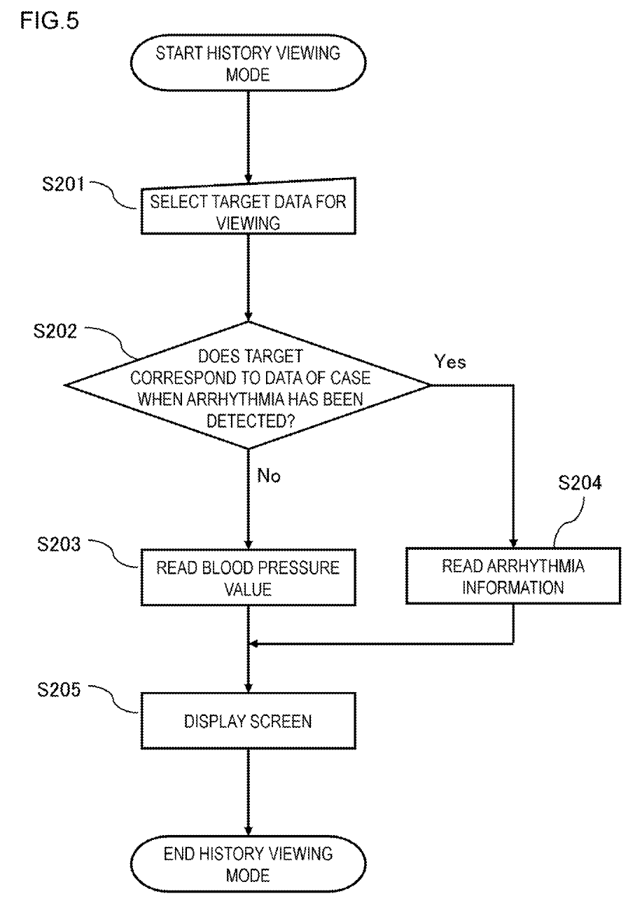

Subsequently, a flow of processing when the past measurement data stored in the storage unit 40 is displayed on the display unit 20 will be described on the basis of FIG. 5. FIG. 5 is a flowchart illustrating a flow of processing (history viewing mode) of referring to past measurement data in the biological information measurement device 1. As illustrated in FIG. 5, first, past measurement data (for example, which is read from the storage unit 40 and displayed as a list on the display unit 20) that the user wants to refer to is selected via the operation unit 30 (S201).

Next, the control unit 100 determines whether or not the measurement data according to the user's selection is data of the case where arrhythmia has been detected during the measurement (S202). Specifically, since the measurement data of the case where arrhythmia has been detected is stored in association with the fact that arrhythmia has been detected and the detail of the arrhythmia, it is determined whether or not the selected measurement data corresponds to such data. In a case where it is determined in step S202 that the measurement data according to the selection is not data of the case where arrhythmia has been detected during the measurement, the control unit 100 reads information on the measured blood pressure value (S203), displays the information on the display unit 20 (S205), and ends the series of processing.

On the other hand, in a case where it is determined in step S202 that the measurement data according to the selection is measurement data of the case where arrhythmia has been detected during the measurement, the control unit 100 reads the information on the arrhythmia (S204), displays a measurement result screen (screen as illustrated in FIG. 4A) indicating the fact that arrhythmia has been present without displaying the blood pressure value on the display unit 20 (S205), and ends the series of processing.

In step S204, in a case where the information on the calculated blood pressure value is also stored together with the information on the arrhythmia in the storage unit 40, only the information on the arrhythmia is selected and read without reading the information on the blood pressure value. On the other hand, when the calculated blood pressure value is discarded and only the information on the arrhythmia is stored in the storage unit 40 at the time of blood pressure measurement, since there is no target blood pressure value in the first place, only the stored information on the arrhythmia is read.

According to such processing, the user can view the past measurement result, and even in a case where arrhythmia has been detected at the time of the past measurement, it is possible to prevent an inaccurate blood pressure measurement result from being indicated to the user, and to notify the user of the arrhythmia detected during the blood pressure measurement.

### Flow of processing in continuous measurement mode

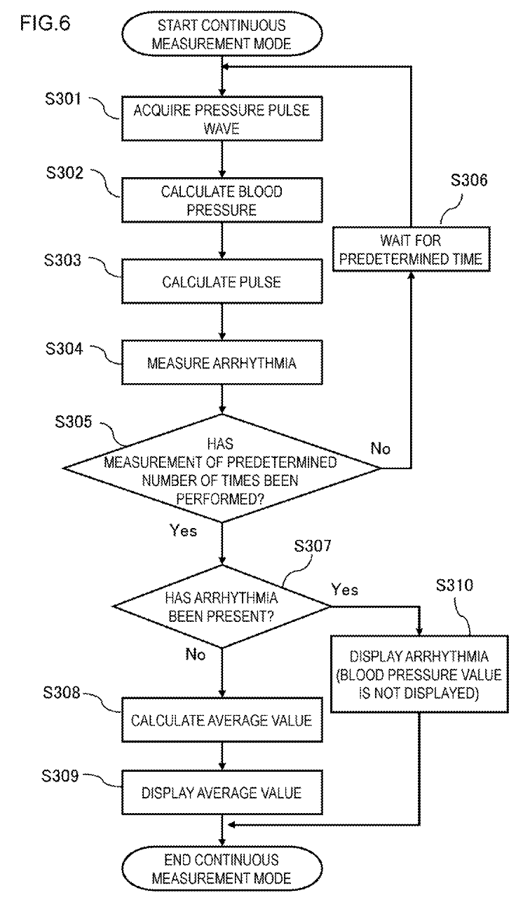

Subsequently, a flow of processing in a case where blood pressure measurement is continuously performed a plurality of times at predetermined intervals will be described on the basis of FIG. 6. FIG. 6 is a flowchart illustrating a flow of processing in a case where the biological information measurement device 1 performs blood pressure measurement in the continuous measurement mode. Prior to the execution of the blood pressure measurement, the user winds the cuff 210 around his/her upper arm, and then instructs execution of the blood pressure measurement in the continuous measurement mode via the operation unit 30.

After receiving the instruction of the blood pressure measurement in the continuous measurement mode, the biological information measurement device 1 acquires the pressure pulse wave of the user by the blood pressure measurement unit 200 (S301). Furthermore, based on the acquired pressure pulse wave, the control unit 100 (the blood pressure value calculation unit 101, the pulse calculation unit 102, and the body motion determination unit 103) detects the body motion of the user (not illustrated), calculates the blood pressure value (S302), and calculates the pulse rate (S303). In addition, the control unit 100 (arrhythmia determination unit 104) determines the presence or absence of arrhythmia during blood pressure measurement on the basis of the acquired pressure pulse wave (S304). Note that the information on the body motion, the blood pressure value, the pulse rate, and the presence or absence of arrhythmia is recorded in the storage unit 40 every time the measurement is performed.

Next, the control unit 100 determines whether or not the blood pressure measurement as one measurement opportunity has been performed a predetermined number of times (S305). In a case where it is determined in step S305 that the measurement for the predetermined number of times has not been performed yet, the control unit 100 waits for a predetermined time (for example, 2 minutes) and then returns to step S301 to repeat the subsequent processing.

On the other hand, in a case where it is determined in step S305 that the measurement for the predetermined number of times has been performed, the control unit 100 continuously determines whether or not arrhythmia has been detected in any of the plurality of times of measurement (S307). In a case where it is determined in step S307 that arrhythmia has not been detected in any of the plurality of measurements, the control unit 100 (one measurement opportunity average value calculation unit 107) calculates an average value of the blood pressure values calculated in the plurality of times of measurement (S308). Then, the control unit 100 (display control unit 105) displays the calculated average value on the display unit 20 (S309), and ends the series of processing.

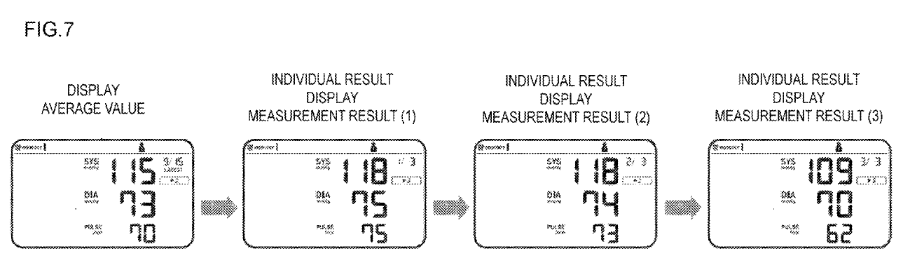

FIG. 7 is an explanatory diagram illustrating a screen display example of the blood pressure measurement result in a case where the measurement has been performed in the continuous measurement mode. The screen of “AVERAGE VALUE DISPLAY” at the left end of FIG. 7 is a screen displaying the measurement result as one measurement opportunity, and is an example of the screen displayed in step S309. In a case where the measurement has been performed in the continuous measurement mode, a user operation allows transition from the average value display to screens respectively showing individual measurement results. As illustrated in FIG. 7, the screen is transitioned from the “AVERAGE VALUE DISPLAY” to the display of the first measurement result, the display of the second measurement result, and the display of the third measurement result in this order to allow viewing of the individual measurement results.

On the other hand, in a case where it is determined in step S307 that arrhythmia has been detected in any of the plurality of measurements, the control unit 100 (display control unit 105) does not display the average value but displays information of the fact that arrhythmia has been present on the display unit 20 (S310), and ends the series of processing.

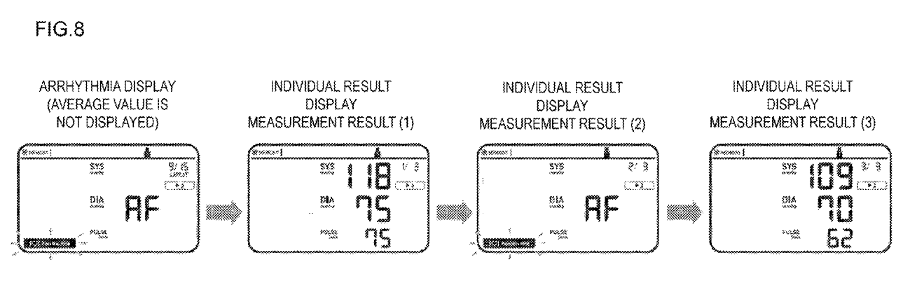

FIG. 8 is an explanatory diagram illustrating a screen display example of the blood pressure measurement result of a case where, when the measurement has been performed in the continuous measurement mode, arrhythmia has been detected at the time of any measurement. The screen of “ARRHYTHMIA DISPLAY” at the left end of FIG. 8 is a screen displaying the measurement result as one measurement opportunity, and is an example of the screen displayed in step S310. Even in a case where arrhythmia has been detected at the time of any measurement, a user operation allows transition to the screens respectively showing individual measurement results. As illustrated in FIG. 8, the screen is transitioned from the “ARRHYTHMIA DISPLAY” to the display of the first measurement result, the display of the second measurement result, and the display of the third measurement result in this order to allow viewing of the individual measurement results.

According to the processing as described above, even in a case where the measurement value of one measurement opportunity is indicated using the average value of measurement values from the plurality of times of measurement, it is possible to prevent an inaccurate blood pressure measurement result from being indicated to the user and to promptly notify the user of arrhythmia.

### Flow of past average value display processing

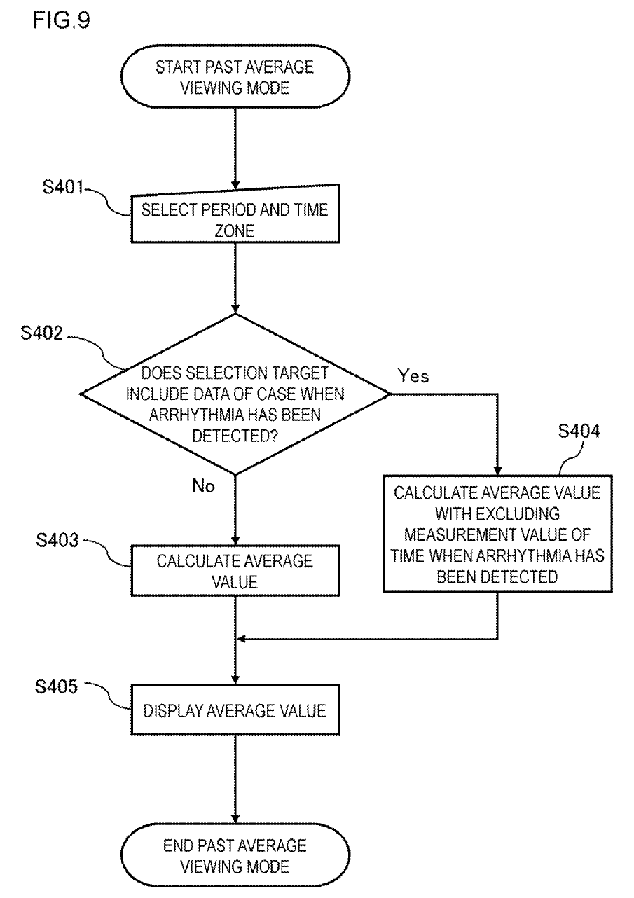

Next, a flow of processing in the biological information measurement device 1 in a case where the average value of the blood pressures in past any period is viewed will be described on the basis of FIG. 9. FIG. 9 is a flowchart illustrating a flow of processing of referring to an average value of the blood pressures measured in past any period (past average viewing mode) by the biological information measurement device 1. As illustrated in FIG. 9, first, a past period and a time zone (for example, morning/night) that the user wants to refer to are selected via the operation unit 30 (S401).

Thereafter, the control unit 100 determines whether or not measurement data for the target period/time zone selected by the user includes data of the case where arrhythmia has been detected (S402). In a case where it is determined in step S402 that the measurement data in a range selected by the user does not include measurement data of the case where arrhythmia has been detected during the measurement, the control unit 100 (past average value calculation unit 108) calculates an average value of a plurality of pieces of measurement data to be selected (S403). Then, the control unit 100 (display control unit 105) displays the calculated average value on the display unit 20, and ends the series of processing.

On the other hand, in a case where it is determined in step S402 that the measurement data in a range selected by the user includes measurement data of the case where arrhythmia has been detected during the measurement, the control unit 100 (past average value calculation unit 108) excludes the measurement data of the case where arrhythmia has been detected to calculate the average value (S404). Then, the control unit 100 (display control unit 105) displays the calculated average value on the display unit 20, and ends the series of processing.

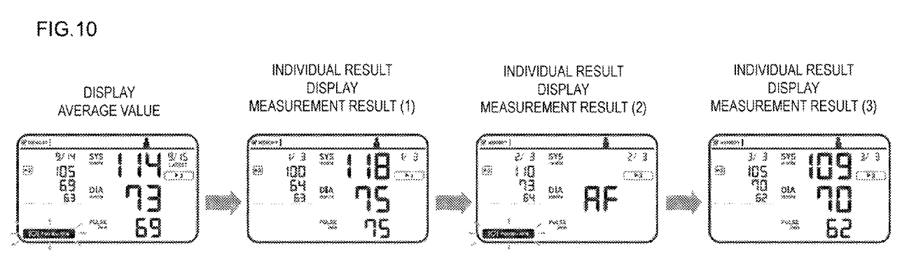

FIG. 10 is an explanatory diagram illustrating a screen display example displayed in a case where, in the past average viewing mode, the measurement data in the range selected by the user includes measurement data of the case where arrhythmia has been detected during the measurement. The screen of “AVERAGE VALUE DISPLAY” at the left end of FIG. 10 is a screen showing the average value calculated by excluding, from the measurement data in the period selected by the user, the measurement data of the case where arrhythmia has been detected. Also in the past average viewing mode, as illustrated in FIG. 10, a user operation allows transition to screens respectively displaying individual measurement results in addition to the display indicating the average value.

According to such processing, the user can view the average value for each time zone in past any period, and it is possible to prevent the average value from being calculated using the inaccurate measurement value of the case where arrhythmia has been detected.

### Modification

In the above embodiment, in a case where arrhythmia has been detected during the blood pressure measurement, the fact that arrhythmia has been detected is displayed without displaying the blood pressure value, thereby preventing the inaccurate blood pressure measurement result from being indicated to the user. However, if the user recognizes that arrhythmia has been detected during the measurement and knows that the measured blood pressure value is inaccurate, the blood pressure value may be displayed as the reference information.

Specifically, for example, the control unit 100 (display control unit 105) performs display as illustrated in FIG. 4B as a measurement result screen of the case where arrhythmia has been detected during the blood pressure measurement. In the screen display example illustrated in FIG. 4B, in the blood pressure display area D1 for displaying the measured blood pressure value, the blood pressure value is not displayed. On the other hand, in an area other than the blood pressure display area D1, the fact that arrhythmia (AFib) has been detected and the fact that the calculated blood pressure value is inaccurate are informed, and operation guidance in the case of viewing the blood pressure value knowing the facts is displayed.

Then, in a case where the decision button is pressed (that is, in a case where the control unit 100 (operation reception unit 106) receives an operation to display the blood pressure value knowing that the value is inaccurate) after the screen as illustrated in FIG. 4B is displayed, control is performed such that the screen transitions to a screen showing the blood pressure value of the case where arrhythmia has been detected during the measurement. As a result, the user can recognize the presence of arrhythmia, and can view the measured blood pressure as the reference value knowing the inaccuracy.

### SECOND EMBODIMENT

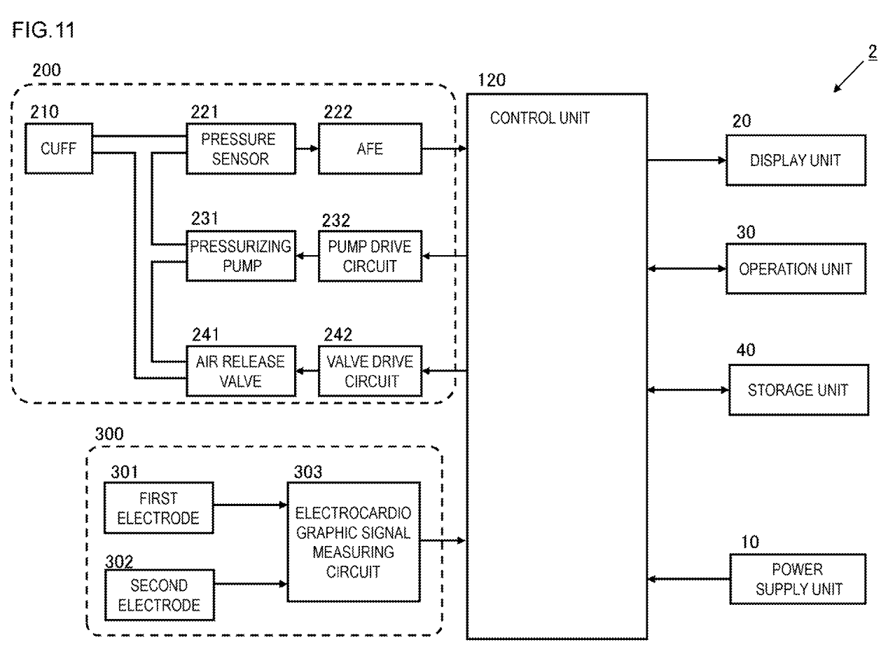

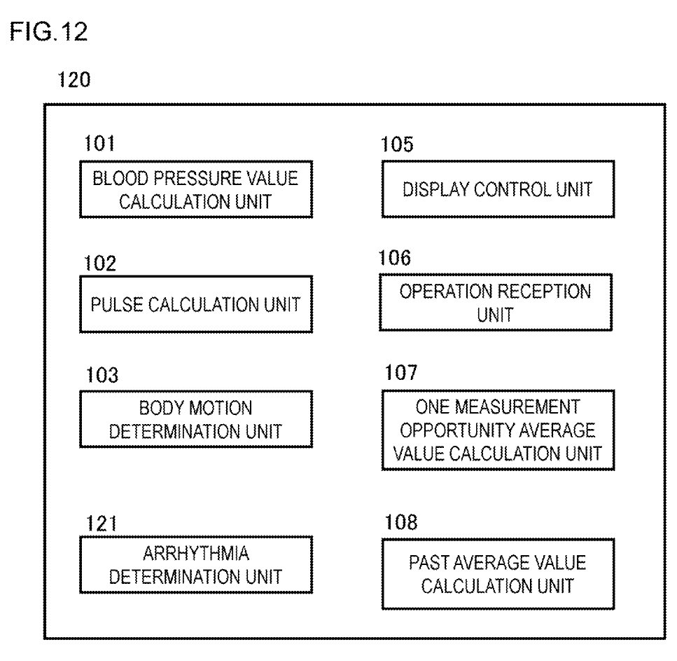

Next, still another embodiment of the present invention will be described on the basis of FIGS. 11 and 12. A biological information measurement device 2 according to the present embodiment is a hybrid biological information measurement device capable of measuring not only blood pressure but also an electrocardiographic waveform. Note that, since the biological information measurement device 2 includes many configurations common to those of the biological information measurement device 1 of the first embodiment, hereinafter, the same reference numerals and signs are used for the same configurations as those of the first embodiment, and repeated description will be omitted.

As illustrated in FIG. 11, the biological information measurement device 2 includes an electrocardiographic waveform measurement unit 300 in addition to the same configurations as those of the biological information measurement device 1. The electrocardiographic waveform measurement unit 300 includes a plurality of electrodes (a first electrode 301 and a second electrode 302) and an electrocardiographic signal measuring circuit 303. The electrocardiographic signal measuring circuit 303 is a circuit for measuring an electrocardiographic waveform on the basis of an electrocardiographic signal obtained on the basis of a potential difference between two electrodes in contact with a living body, and includes an amplifier, a filter, an A/D conversion circuit, and the like.

Although not illustrated, the first electrode 301 and the second electrode 302 are disposed at positions separated from each other in the main body housing. When the user performs measurement processing in a state where one of the left and right fingers is in contact with the first electrode 301 and the other is in contact with the second electrode 302, the potential difference between the first electrode 301 and the second electrode 302 is measured as an electrocardiographic waveform by the electrocardiographic signal measuring circuit 303 (so-called I induction), and the electrocardiographic waveform (electrocardiogram) is measured by recording the potential difference.

Furthermore, as illustrated in FIG. 12, the biological information measurement device 2 includes functional modules including the blood pressure value calculation unit 101, the pulse calculation unit 102, the body motion determination unit 103, the display control unit 105, the one measurement opportunity average value calculation unit 107, the past average value calculation unit 108, and an arrhythmia determination unit 121 which are implemented by a control unit 120.

In the present embodiment, the arrhythmia determination unit 121 detects arrhythmia on the basis of not the pressure pulse wave but the electrocardiographic waveform measured by the electrocardiographic signal measuring circuit 303. Specifically, by simultaneously performing the blood pressure measurement by the blood pressure measurement unit 200 and the electrocardiographic waveform measurement by the electrocardiographic waveform measurement unit 300, the arrhythmia determination unit 121 determines the presence or absence of arrhythmia during the blood pressure measurement on the basis of the electrocardiographic waveform acquired during the blood pressure measurement. That is, in the present embodiment, a combination of the arrhythmia determination unit 121 and the electrocardiographic waveform measurement unit 300 corresponds to the arrhythmia information acquisition unit. Furthermore, the display control unit 105 causes the display unit 20 to display a graph display of the acquired electrocardiographic waveform in response to a user operation.

Note that the flow of processing of the blood pressure measurement in the biological information measurement device 2 according to the present embodiment is similar to that of the biological information measurement device 1 of the first embodiment except that the detection of arrhythmia is performed on the basis of the electrocardiographic waveform instead of the pressure pulse wave, and the display contents of the blood pressure measurement result, the viewing of the past history data of the blood pressure values, and the like are also similar to those of the biological information measurement device 1.

As in the biological information measurement device 2 according to the present embodiment, by detecting arrhythmia on the basis of the electrocardiographic waveform, it is possible to more accurately detect arrhythmia during the blood pressure measurement.

### THIRD EMBODIMENT

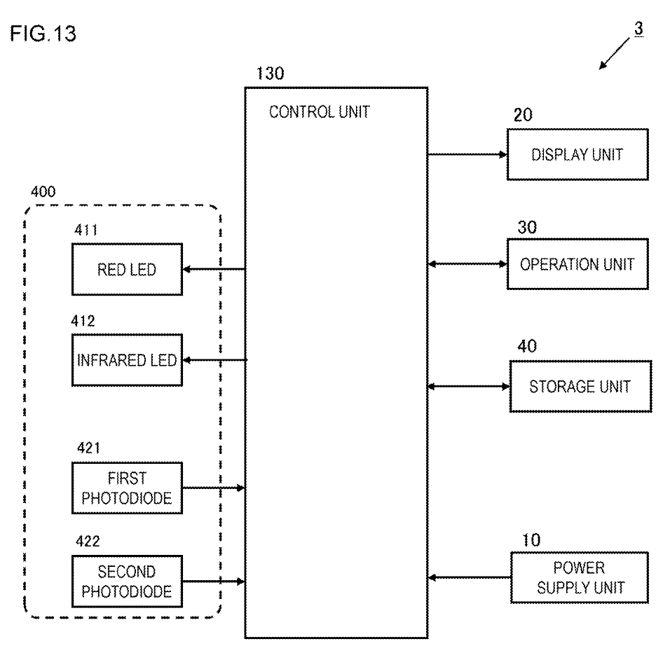

Next, still another embodiment of the present invention will be described on the basis of FIGS. 13 to 16. The biological information measurement device 3 according to the present embodiment is a so-called pulse oximeter capable of measuring a blood oxygen saturation (SpO2) and a pulse rate.

As illustrated in FIG. 13, the biological information measurement device 3 includes configurations of the power supply unit 10, the display unit 20, the operation unit 30, and the storage unit 40 similarly to the biological information measurement device 1, and includes an SpO2 measurement unit 400. Since the configurations of the power supply unit 10, the display unit 20, the operation unit 30, and the storage unit 40 can be similar to those of the biological information measurement device 1 of the first embodiment, a detailed description thereof will be omitted.

The SpO2 measurement unit 400 includes a red LED 411 and an infrared LED 412 as light emitting elements, and a first photodiode 421 and a second photodiode 422 as light receiving elements. The red LED 411, the infrared LED 412, the first photodiode 421, and the second photodiode 422 are all provided so as to be disposed in contact with a surface of a human body (for example, a fingertip of a hand).

The red LED 411 emits red light, and the infrared LED 412 emits infrared light. The first photodiode 421 and the second photodiode 422 receive reflected light of red light and infrared light emitted from the red LED 411 and the infrared LED 412, respectively, and reflected in the living body (more specifically, in a blood vessel), thereby acquiring information for calculating SpO2 (intensity of each reflected light).

Furthermore, the SpO2 measurement unit 400 can not only measure the blood oxygen saturation but also detect a change in the blood flow rate accompanying the heartbeat as a change in the reflected light received by the first photodiode 421 and the second photodiode 422 and acquire the pulse wave (photoelectric pulse wave) on the basis of the change.

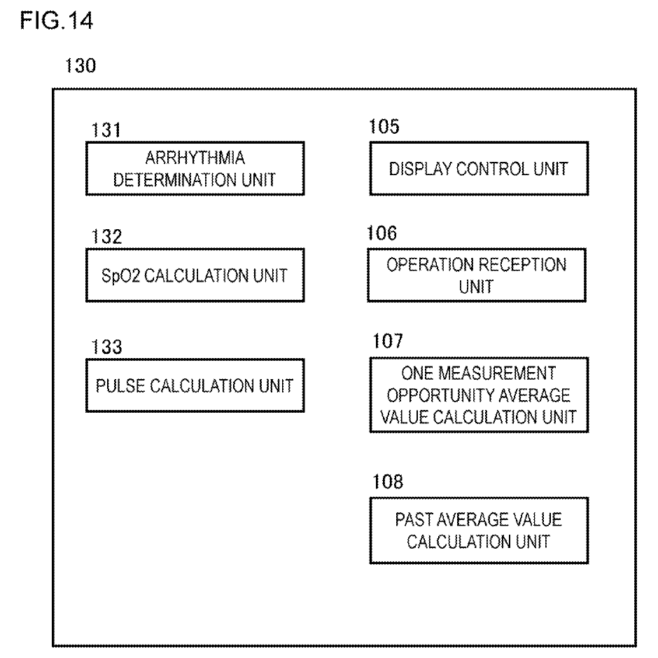

Furthermore, as illustrated in FIG. 14, the biological information measurement device 3 includes functional modules including an arrhythmia determination unit 131, an SpO2 calculation unit 132, a pulse calculation unit 133, the display control unit 105, the operation reception unit 106, the one measurement opportunity average value calculation unit 107, and the past average value calculation unit 108 which are implemented by a control unit 130.

In the present embodiment, the arrhythmia determination unit 131 detects arrhythmia on the basis of not the pressure pulse wave but the photoelectric pulse wave acquired by the SpO2 measurement unit 400. Specifically, the arrhythmia determination unit 131 determines the presence or absence of arrhythmia during biological signal (blood oxygen saturation, pulse) acquisition on the basis of the pulse wave information acquired by the SpO2 measurement unit 400. That is, in the present embodiment, a combination of the arrhythmia determination unit 131 and the SpO2 measurement unit 400 corresponds to the arrhythmia information acquisition unit.

The SpO2 calculation unit 132 calculates the blood oxygen saturation using the information acquired by the SpO2measurement unit 400 (the ratio between the red light and the infrared light received by the photodiodes). Furthermore, the pulse calculation unit 133 calculates the pulse rate on the basis of the pulse wave information acquired by the SpO2 measurement unit 400. Note that the one measurement opportunity average value calculation unit 107 and the past average value calculation unit 108 are similar to those of the biological information measurement device 1 except that the target biological information is not the blood pressure but SpO2 and the pulse rate.

### Flow of processing

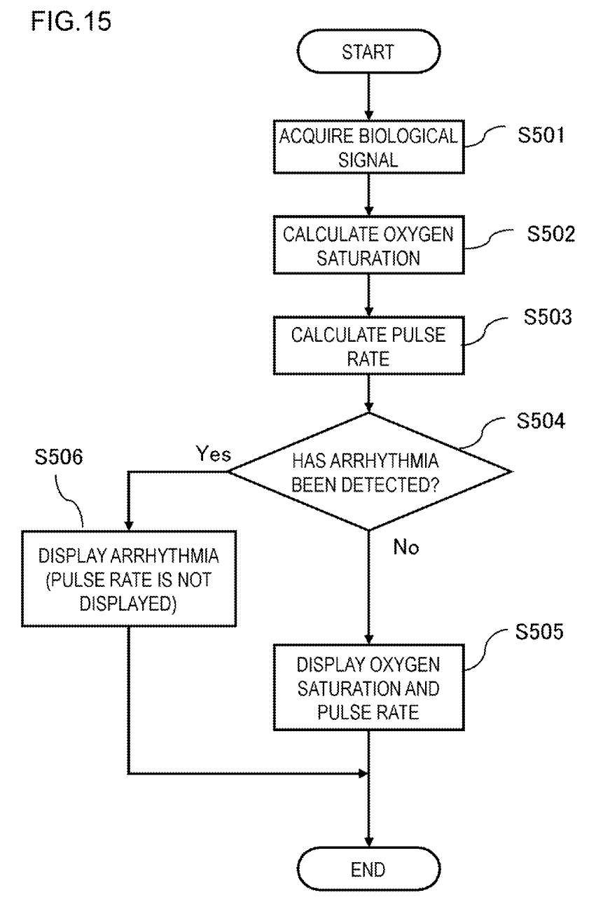

Next, a flow of processing performed by the biological information measurement device 3 according to the present embodiment will be described on the basis of FIG. 15. FIG. 15 is a flowchart illustrating a flow of processing at the time of biological information measurement by the biological information measurement device 3. Prior to the measurement by the biological information measurement device 3, the user wears the biological information measurement device 3 on the finger (for example, the index finger of the right hand), and then instructs execution of SpO2 and pulse measurement via the operation unit 31.

After receiving the measurement instruction, the biological information measurement device 3 acquires the biological information of the user, that is, the red light/infrared light reflected in the blood vessel and the pulse wave (photoelectric pulse wave) by the SpO2measurement unit 400 (S501). Then, based on the acquired information, the control unit 130 (the SpO2 calculation unit 132 and the pulse calculation unit 133) calculates the blood oxygen saturation (S502), and calculates the pulse rate (S503). In addition, the control unit 130 (arrhythmia determination unit 131) determines the presence or absence of arrhythmia during pulse wave acquisition on the basis of the acquired pulse wave (S504).

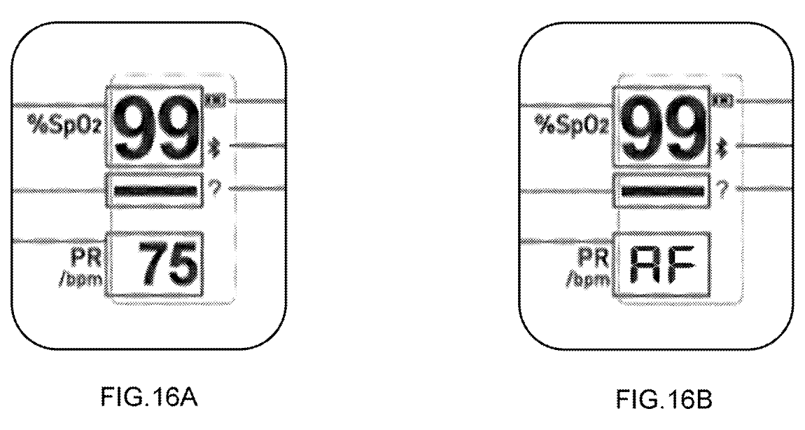

In a case where it is determined in step S504 that arrhythmia has been absent during pulse wave acquisition, the control unit 130 (display control unit 105) displays the blood oxygen saturation calculated in step S502 and the pulse rate calculated in step S503 on the display unit 20 (S505). FIG. 16A is a diagram illustrating an example of the measurement result screen displayed on the display unit 20 in step S505.

On the other hand, in a case where it is determined in step S504 that arrhythmia has been present during the pulse wave acquisition, the control unit 130 (display control unit 105) does not display the calculated pulse rate but displays, as measurement results, the measurement value of the blood oxygen saturation and the fact that arrhythmia has been present on the display unit 20 (S506). FIG. 16B is a diagram illustrating an example of a screen displayed on the display unit 20 in a case where arrhythmia has been detected during the pulse wave acquisition in this manner. In the example illustrated in FIG. 16B, in the area for displaying the measured pulse rate of the display unit 20, the pulse rate is not displayed, but “AF” indicating that arrhythmia (in this case, atrial fibrillation) has been detected is displayed.

After displaying the measurement result on the display unit 20 in step S505 or step S506, the biological information measurement device 3 stores the measurement result in the storage unit 40 in association with the information on the measurement time, and ends the series of processing. Note that the biological information measurement device 3 is similar to the biological information measurement device 1 of the first embodiment in that, when the past measurement result (history data) stored in the storage unit 40 is viewed, the pulse rate is not displayed in a case where the past measurement result related to the viewing is the result of the case where arrhythmia has been detected. In addition, calculation and display of the average value from the storage unit can also be performed similarly to the biological information measurement device 1 of the first embodiment.

### FOURTH EMBODIMENT

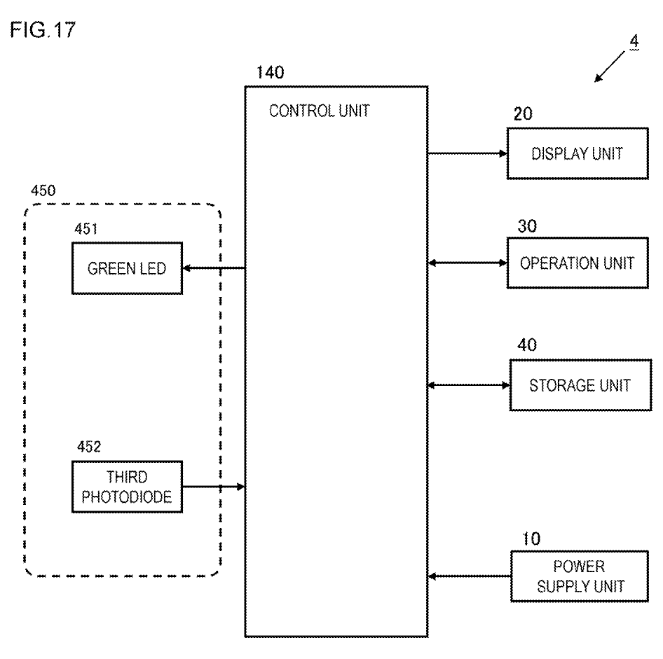

Next, a fourth embodiment of the present invention will be described on the basis of FIGS. 17 to 20. A biological information measurement device 4 according to the present embodiment is a pulsometer capable of measuring a pulse, and can be, for example, a wristwatch-type wearable device.

As illustrated in FIG. 17, the biological information measurement device 4 includes configurations of the power supply unit 10, the display unit 20, the operation unit 30, and the storage unit 40 similarly to the biological information measurement device 1, and includes a photoelectric pulse wave measurement unit 450. Since the configurations of the power supply unit 10, the display unit 20, the operation unit 30, and the storage unit 40 can be similar to those of the biological information measurement device 1 of the first embodiment, a detailed description thereof will be omitted.

The photoelectric pulse wave measurement unit 450 includes a green LED 451 as a light emitting element, and a third photodiode 452 as a light receiving element. Both the green LED 451 and the third photodiode 452 are provided so as to be disposed in contact with a surface of a human body (for example, a wrist).

The green LED 451 emits green light, and the third photodiode 452 receives reflected light emitted from the green LED 451 and reflected in a living body (more specifically, in a blood vessel), thereby detecting a blood flow rate (change in volume of the blood vessel) that changes with the heartbeat of the heart and measuring a pulse wave.

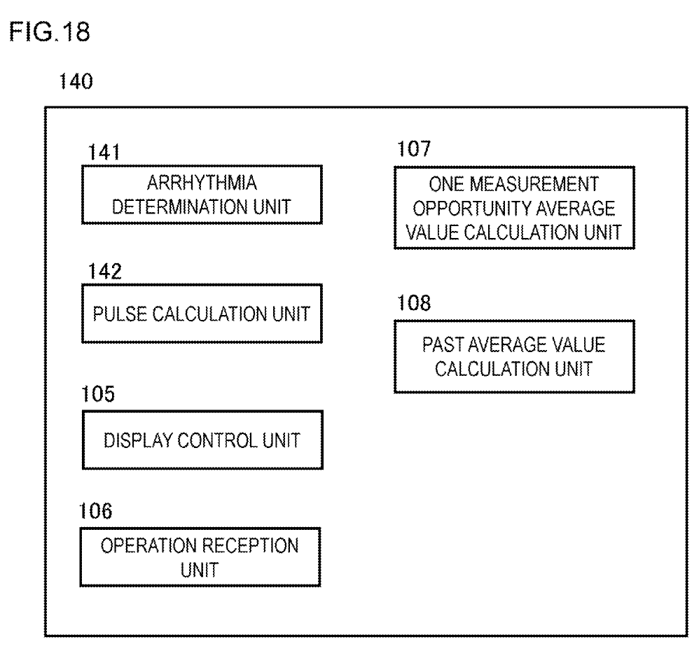

Furthermore, as illustrated in FIG. 18, the biological information measurement device 4 includes functional modules including an arrhythmia determination unit 141, a pulse calculation unit 142, the display control unit 105, the operation reception unit 106, the one measurement opportunity average value calculation unit 107, and the past average value calculation unit 108 which are implemented by a control unit 140.

In the present embodiment, the arrhythmia determination unit 141 detects arrhythmia on the basis of the photoelectric pulse wave acquired by the photoelectric pulse wave measurement unit 450. That is, in the present embodiment, a combination of the arrhythmia determination unit 141 and the photoelectric pulse wave measurement unit 450 corresponds to the arrhythmia information acquisition unit.

The pulse calculation unit 142 calculates the pulse rate on the basis of the pulse wave information acquired by the photoelectric pulse wave measurement unit 450. Note that the one measurement opportunity average value calculation unit 107 and the past average value calculation unit 108 are similar to those of the biological information measurement device 1 except that the target biological information is not the blood pressure but the pulse rate.

### Flow of processing

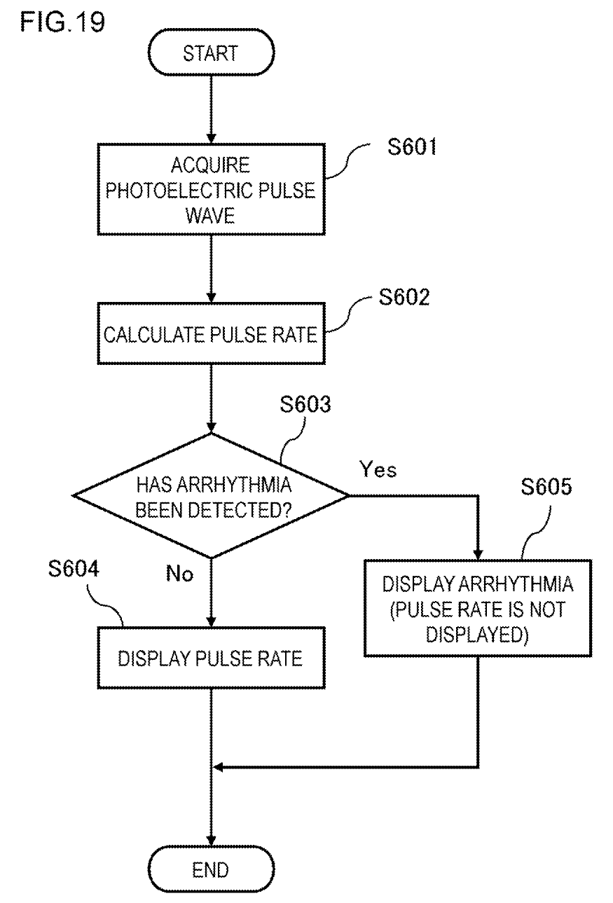

Next, a flow of processing performed by the biological information measurement device 4 according to the present embodiment will be described on the basis of FIG. 19. FIG. 19 is a flowchart illustrating a flow of processing at the time of biological information measurement by the biological information measurement device 4. Prior to the measurement by the biological information measurement device 4, the user wears the biological information measurement device 4 on the wrist or the like, and then instructs execution of pulse measurement via the operation unit 30.

After receiving the instruction of the measurement, the biological information measurement device 4 acquires the biological information (that is, pulse wave) of the user by the photoelectric pulse wave measurement unit 450 (S601). Then, based on the acquired information, the control unit 140 (pulse calculation unit 142) calculates the pulse rate (S603). In addition, the control unit 140 (arrhythmia determination unit 141) determines the presence or absence of arrhythmia during pulse wave acquisition on the basis of the acquired pulse wave (S603).

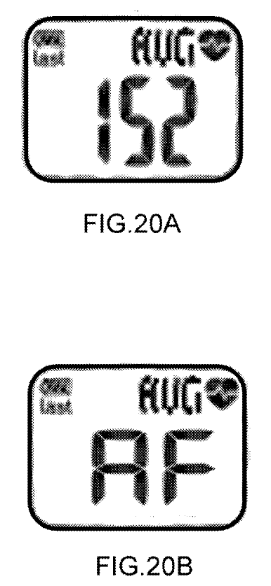

In a case where it is determined in step S603 that arrhythmia has been absent during pulse wave acquisition, the control unit 140 (display control unit 105) displays the pulse rate calculated in step S602 on the display unit 20 (S604). FIG. 20A is a diagram illustrating an example of the measurement result screen displayed on the display unit 20 in step S604.

On the other hand, in a case where it is determined in step S603 that arrhythmia has been present during the pulse wave acquisition, the control unit 140 (display control unit 105) does not display the calculated pulse rate but displays, as a measurement result, the fact that arrhythmia has been present on the display unit 20 (S605). FIG. 20B is a diagram illustrating an example of a screen displayed on the display unit 20 in a case where arrhythmia has been detected during the pulse wave acquisition in this manner. In the example illustrated in FIG. 20B, in the area for displaying the measured pulse rate of the display unit 20, the pulse rate is not displayed, but “AF” indicating that arrhythmia (in this case, atrial fibrillation) has been detected is displayed.

After displaying the measurement result on the display unit 20 in step S604 or step S605, the biological information measurement device 4 stores the measurement result in the storage unit 40 in association with the information on the measurement time, and ends the series of processing. Note that the biological information measurement device 3 is similar to the biological information measurement device 1 of the first embodiment in that, when the past measurement result (history data) stored in the storage unit 40 is viewed, the pulse rate is not displayed in a case where the past measurement result related to the viewing is the result of the case where arrhythmia has been detected. In addition, calculation and display of the average value from the storage unit can also be performed similarly to the biological information measurement device 1 of the first embodiment.

### FIFTH EMBODIMENT

### System configuration

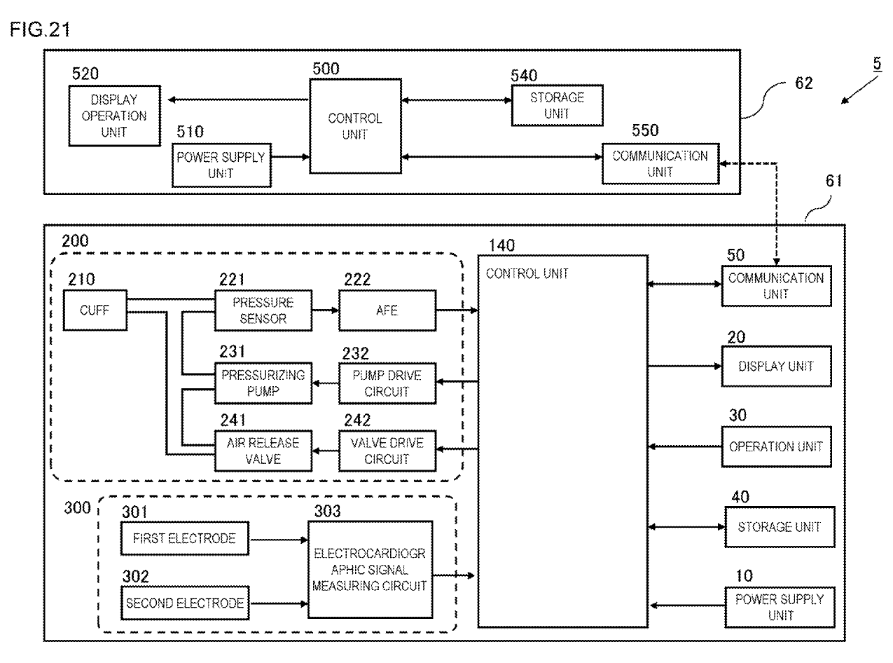

In each of the above embodiments, the detection of arrhythmia during the biological signal acquisition (determination of the presence or absence of arrhythmia) is executed in the biological information measurement device, but the detection of arrhythmia during the biological signal acquisition may be executed in another device. That is, the present invention is also applicable as a system including a biological information measurement device and an information processing terminal as in an embodiment described below. FIG. 21 is a block diagram schematically illustrating a configuration of an information processing system 5 according to the present embodiment.

As illustrated in FIG. 21, the information processing system 5 according to the present embodiment includes a biological information measurement device 61 including the blood pressure measurement unit 200 and the electrocardiographic waveform measurement unit 300, and a smartphone 62 as an example of the information processing terminal, which are configured to be communicably connectable.

The biological information measurement device 61 has a hardware configuration substantially similar to that of the biological information measurement device 2 according to the second embodiment, and is only different in including a communication unit 50. The communication unit 50 functions to communicate with and connect to the smartphone 62, and can adopt an appropriate communication interface according to a communication network to be connected, such as a wired communication port or a wireless communication antenna. Note that a communication method between the biological information measurement device 61 and the smartphone 62 is not particularly limited, and for example, pairing by Bluetooth (registered trademark) low energy (BLE) communication, communication by Wi-Fi (registered trademark) (LAN), or the like can be adopted.

As illustrated in FIG. 21, the smartphone 62 includes configurations such as a power supply unit 510, a display operation unit 520, a storage unit 540, a communication unit 550, and a control unit 500.

The power supply unit 510 is a power source of the smartphone 62, and can be, for example, a rechargeable secondary battery such as a lithium ion battery. The display operation unit 520 can be a touch panel display or the like having both functions of information display and input operation reception.

The storage unit 540 includes a main storage device such as a RAM and a ROM, and an auxiliary storage device such as a flash memory, and stores various types of information such as an application program and various pieces of biological information. Note that the measurement results of the blood pressure and the like and other acquired biological information may be stored in the storage unit 540 in association with time information such as the measurement (acquisition) time. As the time information, for example, time information clocked by referring to an RTC of the control unit 500 can be used. Furthermore, the auxiliary storage device may be configured to be detachable from the main body housing (not illustrated).

The communication unit 550 functions to communicate with and connect to the biological information measurement device 61, and can adopt an appropriate communication interface according to a communication network to be connected, such as a wired communication port or a wireless communication antenna.

The control unit 500 is a functional unit that controls the smartphone, and includes, for example, a processor such as a CPU and a GPU. When receiving a user's operation via the display operation unit 520, the control unit 500 controls each component of the smartphone to exhibit various functions according to a predetermined application program. Note that the predetermined program is stored in the storage unit 540 and read therefrom.

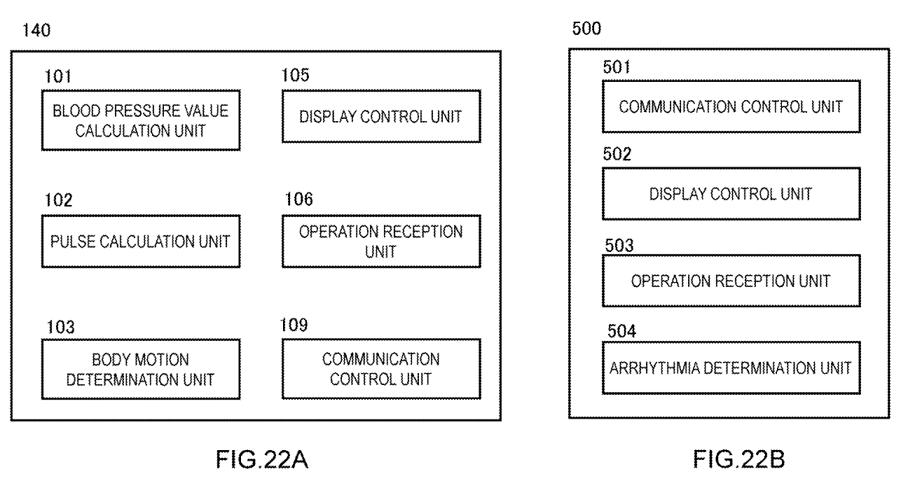

FIG. 22A is a block diagram illustrating part of functional modules implemented by the control unit 140 of the biological information measurement device 61, and FIG. 22B is a block diagram illustrating part of functional modules implemented by the control unit 500 of the smartphone 62.

As illustrated in FIG. 22A, the functional modules of the biological information measurement device 61 are different from those of the biological information measurement device 2 of the second embodiment in that a communication control unit 109 is added and the arrhythmia determination unit does not exist. The communication control unit 109 executes communication with an external device (for example, the smartphone 62) by the communication unit 50.

As illustrated in FIG. 22B, the control unit 500 of the smartphone 62 implements functional modules including a communication control unit 501, a display control unit 502, and an arrhythmia determination unit 504. The communication control unit 501 executes communication with an external device (for example, the biological information measurement device 61) by the communication unit 550.

The display control unit 502 controls the display contents of the display operation unit 520. For example, the display control unit 502 causes the display operation unit 520 to display a menu screen (operation reception screen), guidance related to the progress of biological information measurement, a measurement result of biological information, and the like according to an application program related to cooperation with the biological information measurement device 61.

The operation reception unit 503 receives an input of a user operation via a user interface displayed on the display operation unit 520. For example, in accordance with an application program related to cooperation with the biological information measurement device 61, the operation reception unit 503 receives an operation input such as start of measurement, selection of an item, and screen transition by the biological information measurement device 61.

As will be described later, the arrhythmia determination unit 504 determines (detects), on the basis of the information on the electrocardiographic waveform transmitted from the biological information measurement device 61, the presence or absence of arrhythmia at the time of blood pressure measurement performed simultaneously with the acquisition of the electrocardiographic waveform. That is, in the present embodiment, the detection processing of arrhythmia during the blood pressure measurement is performed not by the biological information measurement device 61 but by the smartphone 62. Then, the information on the presence or absence of arrhythmia determined by the arrhythmia determination unit 504 is transmitted from the communication unit 550 and received by the communication unit 50 of the biological information measurement device 61. That is, in the biological information measurement device 61 in the present embodiment, the communication unit 50 corresponds to the arrhythmia information acquisition unit.

### Flow of processing

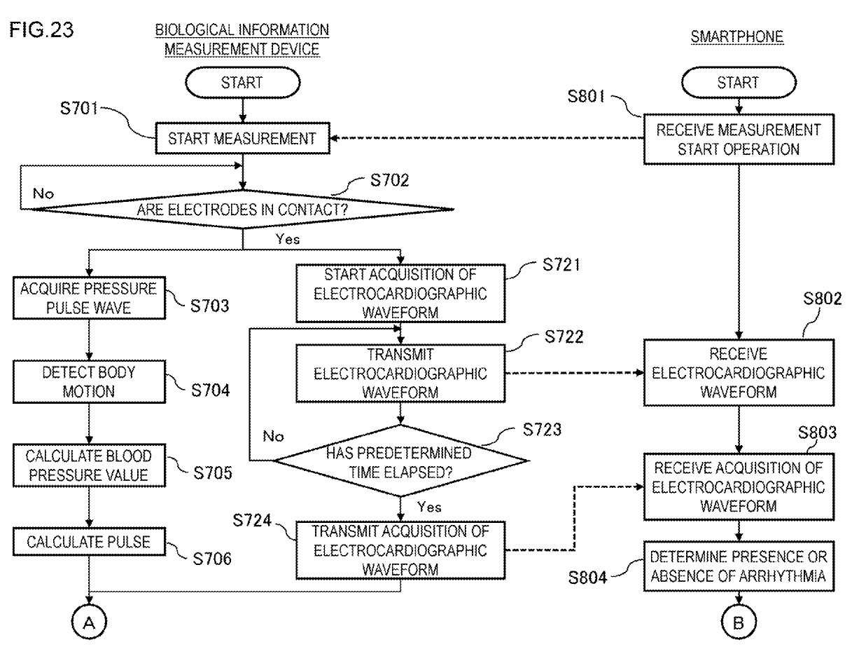

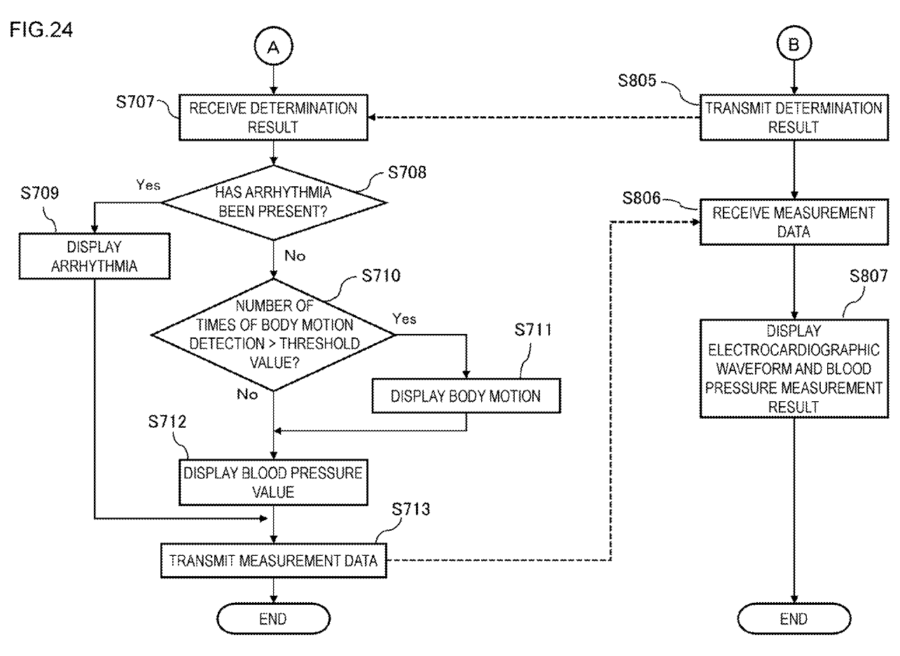

Subsequently, based on FIGS. 23 and 24, flows of blood pressure/electrocardiogram collective measurement processing executed in the information processing system 5 according to the present embodiment and information will be described. In the information processing system 5 according to the present embodiment, the smartphone 62 can instruct to start the collective measurement of the blood pressure and the electrocardiographic waveform. Therefore, the control unit 500 (operation reception unit 503) of the smartphone 62 receives the operation input of starting the collective measurement of the blood pressure and the electrocardiogram via the display operation unit 520 (S801). The instruction by the operation input is transmitted from the smartphone 62 to the biological information measurement device 61 via the communication unit 550, and the biological information measurement device 61 starts the collective measurement of the blood pressure and the electrocardiogram (S701).

When the collective measurement is started, the biological information measurement device 61 determines whether or not the first electrode 201 and the second electrode 202 are correctly in contact with the living body on the basis of the signal acquired by the electrocardiographic waveform measurement unit 300 (S702). Here, in a case where it is determined that both electrodes are not correctly in contact with the living body, the processing to determine the electrode contact is repeatedly performed. On the other hand, in a case where it is determined in step S702 that both electrodes are correctly in contact with the living body, the biological information measurement device 61 executes acquisition of the pressure pulse wave by the blood pressure measurement unit 200 and acquisition of the electrocardiographic waveform by the electrocardiographic waveform measurement unit 300 in parallel (S703, S721).

The electrocardiographic waveform acquired in step S721 by the biological information measurement device 61 is transmitted to the smartphone 62 via the communication unit 50 (S722), and the smartphone 62 receives the electrocardiographic waveform (S802). The biological information measurement device 61 (control unit 140) continues to acquire the electrocardiographic waveform, and determines whether or not a predetermined time (for example, time sufficient for pulse wave acquisition for blood pressure measurement) has elapsed (S723). Here, in a case where it is determined that the predetermined time has not elapsed, the process returns to step S722, and the subsequent processing is repeated.

On the other hand, in a case where it is determined in step S723 that the predetermined time has elapsed, the biological information measurement device 61 ends the acquisition of the electrocardiographic waveform and transmits that fact to the smartphone 62 via the communication unit 50 (S724), and the smartphone 62 receives the information of the fact that the acquisition of the electrocardiographic waveform has ended (S803). Then, in the smartphone 62 that has received the electrocardiographic waveform for the predetermined time, the control unit 500 (arrhythmia determination unit 504) executes analysis processing of determining (detecting) the presence or absence of arrhythmia during the blood pressure measurement (during pressure pulse wave acquisition) on the basis of the electrocardiographic waveform (S804).

On the other hand, in the biological information measurement device 61, in parallel with the acquisition of the electrocardiographic waveform, acquisition of a pressure pulse wave for blood pressure measurement is performed by the blood pressure measurement unit 200 (S703). In addition, based on the acquired pressure pulse wave, the control unit 140 (the blood pressure value calculation unit 101, the pulse calculation unit 102, and the body motion determination unit 103) detects the body motion of the user (S704), calculates the blood pressure value (S705), and calculates the pulse rate (S706).

Next, referring to FIG. 24, the smartphone 62 transmits the determination result of the presence or absence of arrhythmia performed in step S804 to the biological information measurement device 61 via the communication unit 550 (S805), and the biological information measurement device 61 receives the determination result (S707). Then, in a case where the determination result received in step S707 indicates the presence of arrhythmia during the blood pressure measurement, the biological information measurement device 61 (display control unit 105) does not display the calculated blood pressure value but displays, as a measurement result, the fact that arrhythmia has been present on the display unit 20 (S708, S709).

On the other hand, in a case where the received determination result is the absence of arrhythmia during the blood pressure measurement, the biological information measurement device 61 (body motion determination unit 103) subsequently determines whether or not the number of times of body motion detected during the blood pressure measurement exceeds a predetermined threshold value (S708, S710). Then, in a case where it is determined in step S710 that the number of times of body motion is equal to or less than the threshold value, the biological information measurement device 61 (display control unit 105) displays the blood pressure value and the pulse rate on the display unit 20 (S712).

In a case where it is determined in step S710 that the number of times of body motion exceeds the threshold value, the biological information measurement device 61 (display control unit 105) displays the fact that body motion has been detected in an area other than the blood pressure display area D1 of the display unit 20 (S711), and displays the blood pressure value in the blood pressure display area D1 (S712). Note that step S711 and step S712 may be executed simultaneously.

Thereafter, the biological information measurement device 61 transmits the calculated blood pressure value, pulse rate, and number of times of body motion detection (hereinafter, collectively referred to as blood pressure measurement data) to the smartphone 62 via the communication unit 50 (S713), and ends the series of processing. On the other hand, the smartphone 62 receives the blood pressure measurement data (S806), displays the blood pressure measurement data and the electrocardiographic waveform (electrocardiogram) on the display operation unit 520 (S807), and ends the series of processing.

Note that the order of the processing described above can be appropriately changed or omitted. For example, the transmission of the blood pressure measurement data from the biological information measurement device 61 is not limited to after step S712, and may be executed at other timings such as after step S707. Furthermore, the smartphone 62 may end the series of processing after step S805 without transmitting the blood pressure measurement data from the biological information measurement device 61.

Furthermore, since it is undetermined which of the processing of the blood pressure value calculation and the processing of the arrhythmia presence or absence determination ends first, step S707 (and step S805) may be executed before the blood pressure value calculation in step S705.

In the above example, in step S723, the biological information measurement device 61 determines whether or not the predetermined time (for example, time sufficient for pulse wave acquisition for blood pressure measurement) has elapsed from the start of acquisition of the electrocardiographic waveform. However, this processing may be executed on the smartphone 62 side. In this case, the smartphone 62 may transmit a command to end the acquisition of the electrocardiographic waveform to the biological information measurement device 61 after the predetermined time elapses.

In the smartphone 62, after the electrocardiographic waveform is received in step S802, the electrocardiographic waveform may be displayed on the display operation unit 520 in real time.

According to the information processing system 5 as described above, the determination of the presence or absence of arrhythmia during the blood pressure measurement can be executed by the information processing terminal such as the smartphone 62 instead of the biological information measurement device 61. As a result, the calculation load of the biological information measurement device can be reduced, and even if a high-performance calculation device is not used, it is possible to prevent an inaccurate blood pressure measurement result from being indicated to the user and to notify the user of the arrhythmia detected during the blood pressure measurement.

### Modification

In the biological information processing system 5, in a case where arrhythmia has been detected during the blood pressure measurement, the fact that arrhythmia has been detected is displayed on the display unit 20 or the display operation unit 520 without displaying the blood pressure value. However, similarly to the modification of the first embodiment, it is possible to adopt the configuration in which the measured blood pressure value is indicated as the reference value.

For example, after the display unit 20 of the biological information measurement device 61 displays in step S709 the fact that arrhythmia is present, the display control unit 502 of the smartphone 62 causes the display operation unit 520 to display the fact that arrhythmia has been detected during the blood pressure measurement, the fact that the measured blood pressure value is inaccurate, and an interface (button icon) indicating whether or not to display the measured blood pressure value as the reference value knowing the facts. Then, when the operation reception unit 503 receives a user operation to display the blood pressure value, the operation reception unit 503 may transmit a signal to display the blood pressure value to the biological information measurement device 61 so that the biological information measurement device 61 displays the blood pressure value on the display unit 20.

Similarly, in the smartphone 62, in a case where arrhythmia has been present during the measurement, the blood pressure value is not displayed in step S807, the fact that arrhythmia has been detected during the blood pressure measurement, the fact that the measured blood pressure value is inaccurate, and an interface indicating whether or not to display the measured blood pressure value as the reference value knowing the facts are displayed, and the blood pressure value may be displayed after the display permission operation is performed.

### OTHERS

Note that the description of the above-described embodiments is merely illustrative of the present invention, and the present invention is not limited to the above-described specific forms. The present invention can be variously modified and combined within the scope of the technical idea. For example, the configuration according to the second embodiment and the configuration according to the third embodiment may be combined to form a biological information measurement device including all of the blood pressure measurement unit 200, the electrocardiographic waveform measurement unit 300, and the SpO2 measurement unit 400.

In the above embodiments, when the fact that arrhythmia has been detected during the biological signal acquisition is displayed, the display mode may be made different from the display mode at the time of notifying of another measurement error or failure of the device. Specifically, the detection of arrhythmia can be clearly identified from other errors by a difference in lighting/blinking of the display, a difference in blinking interval, a difference in display color, and the like.

In the second embodiment, the electrocardiographic waveform measurement unit 300 is exemplified as the device integrated with the blood pressure measurement unit 200. However, the present invention is not limited thereto, and a configuration can be adopted in which the electrocardiographic waveform or photoelectric pulse wave acquired by a separate measuring instrument is acquired via the communication unit. In this case, the communication unit serves as the arrhythmia information acquisition unit.

In the fifth embodiment, the biological information measurement device 61 includes the blood pressure measurement unit 200 and the electrocardiographic waveform measurement unit 300. However, the technology of determining the presence or absence of arrhythmia during the biological signal acquisition using another information processing terminal is naturally applicable to other biological information measurement devices as described in the first, third, and fourth embodiments. Furthermore, the information processing terminal is not limited to the smartphone 62, and other information processing device can be adopted regardless of whether the information processing device is a portable type or a stationary type.

In each of the above examples, the description has been made using the atrial fibrillation as an example of arrhythmia. However, as a matter of course, also in a case where another arrhythmia (atrial flutter, premature contraction, etc.) has been detected, the fact that the atrial flutter, the premature contraction, or the like has been detected may be displayed without displaying the blood pressure value similarly to the above embodiments.

What is claimed is:

1. A biological information measurement device that acquires a biological signal and calculates a measurement value for a predetermined index related to the biological signal on the basis of the biological signal acquired, the biological information measurement device comprising:
an arrhythmia information acquisition unit that acquires information related to presence or absence of arrhythmia in a living body during acquisition of the biological signal;
a display unit including an area capable of displaying at least the measurement value;
an input unit that receives an operation input by a user; and
a control unit, wherein
the control unit
calculates the measurement value on the basis of the biological signal acquired, and
causes, in a case where the information acquired by the arrhythmia information acquisition unit indicates that arrhythmia during the acquisition of the biological signal has been present, the display unit not to display the measurement value calculated but to display information of a fact that arrhythmia has been present.

2. The biological information measurement device according to claim 1, further comprising
a storage unit that holds information related to calculation of the measurement value performed in a past, the information including information related to a date and time of the calculation and presence or absence of arrhythmia during acquisition of the biological signal at the date and time, wherein
the control unit
receives, via the input unit, a request to display, on the display unit, the information related to the measurement value of the past held in the storage unit, and
causes, in a case where the information related to the measurement value related to the request to display includes information of the fact that arrhythmia has been present during the acquisition of the biological signal related to the calculation of the measurement value, the display unit not to display the measurement value of the past but to display the information of the fact that arrhythmia has been present during the acquisition of the biological signal.

3. The biological information measurement device according to claim 1, wherein
the control unit
calculates a first average value that is an average value of the measurement values in a case where the calculation of the measurement value is performed a plurality of times at predetermined intervals according to an operation by a user, and causes the display unit to display the first average value, and
causes, in a case where the information acquired by the arrhythmia information acquisition unit indicates that arrhythmia has been present during the acquisition of the biological signal in any of the plurality of calculations of the measurement value, the display unit not to display the first average value but to display information of the fact that arrhythmia has been present.

4. The biological information measurement device according to claim 2, wherein
the control unit
receives, via the input unit, a request to calculate a second average value that is an average value in past any period using the information related to the calculation of the measurement value held in the storage unit,
excludes, in a case where any of a plurality of pieces of the information related to the calculation of the measurement value related to the request to calculate includes information of the fact that arrhythmia has been present during the acquisition of the biological signal, the information related to the calculation of the measurement value including the information of the fact that arrhythmia has been present to calculate the second average value, and
causes the display unit to display the second average value calculated.

5. The biological information measurement device according to claim 1, further comprising
an electrocardiographic waveform acquisition unit that acquires an electrocardiographic waveform of the living body, wherein
the arrhythmia information acquisition unit acquires the information related to presence or absence of arrhythmia in the living body on the basis of the electrocardiographic waveform.

6. The biological information measurement device according to claim 1, wherein
the control unit
causes, in a case where a user operation to display a measurement value calculated when arrhythmia has been present is received via the input unit after causing the display unit not to display the measurement value but to display information of the fact that arrhythmia has been present, the display unit to display the measurement value.

7. A control method of a biological information measurement device, the control method comprising:
acquiring a biological signal;
calculating a measurement value for a predetermined index related to the biological signal on the basis of the biological signal;
determining presence or absence of arrhythmia in the living body during acquisition of the biological signal; and
displaying a measurement result related to the index, wherein,
in a case where it is determined, in the determining presence or absence of arrhythmia, that arrhythmia has been present, a value of the measurement value calculated is not displayed but information of a fact that arrhythmia has been present is displayed in the displaying the measurement result.

8. A non-transitory computer-readable storage medium recording a program for causing a biological information measurement device to execute the acquiring, the calculating, the determining, and the displaying according to claim 7.

9. An information processing system comprising:
a biological information measurement device that acquires a biological signal of a living body and calculates a measurement value for a predetermined index related to the biological signal on the basis of the biological signal acquired; and an information processing terminal communicatively connected to the biological information measurement device, wherein
the information processing terminal
receives, from the biological information measurement device, at least determination information for determining presence or absence of arrhythmia in the living body during acquisition of the biological signal,
determines presence or absence of arrhythmia during the acquisition of the biological signal on the basis of the determination information, and
transmits, in a case where it is determined that arrhythmia has been present during the acquisition of the biological signal, information of a fact that arrhythmia has been present during the acquisition of the biological signal to the biological information measurement device, and wherein
the biological information measurement device includes a first display unit, and
causes, in a case where the information of the fact that arrhythmia has been present during the acquisition of the biological signal is received from the information processing terminal, the first display unit not to display the measurement value calculated but to display the information of the fact that arrhythmia has been present.

10. The information processing system according to claim 9, wherein
the biological information measurement device includes an electrocardiographic waveform acquisition unit that acquires an electrocardiographic waveform of the living body, and
the determination information is the electrocardiographic waveform.

11. The information processing system according to claim 9, wherein
the information processing terminal includes a second display unit,
receives the measurement value calculated by the biological information measurement device,
displays the measurement value received on the second display unit in a case where it is determined that arrhythmia has been absent during the acquisition of the biological signal, and does not display the measurement value but displays the information of the fact that arrhythmia has been present on the second display unit in a case where it is determined that arrhythmia has been present during the acquisition of the biological signal.

12. The information processing system according to claim 9, wherein
the information processing terminal
includes a second display unit,
receives the measurement value calculated by the biological information measurement device,
displays the measurement value received on the second display unit in a case where it is determined that arrhythmia has been absent during the acquisition of the biological signal, and does not display the measurement value but displays the information of the fact that arrhythmia has been present on the second display unit in a case where it is determined that arrhythmia has been present during the acquisition of the biological signal, and
displays, in a case where a user operation to display the measurement value calculated when the arrhythmia has been present is received after causing the second display unit to display the information of the fact that arrhythmia has been present, the measurement value on the second display unit.

13. A non-transitory computer-readable storage medium recording a program for causing an information processing device to function as the information processing terminal according to claim 9.
